(19) United States
(12) Patent Application Publication
(10) Pub. No.: US 2019/0083619 A1
(43) Pub. Date: Mar. 21, 2019

(54) METHODS AND COMPOSITIONS FOR REMOVING, REDUCING, DISPERSING, DISRUPTING OR ERADICATING BIOFILMS AND DETERMINING THE NUMBER OF BACTERIAL POPULATION IN BIOFILM OR A CULTURE CONTAINING BACTERIAL AGGREGATE

(71) Applicant: INSTITUTE OF MICROBIOLOGY, CHINESE ACADEMY OF SCIENCES, Beijing (CN)

(72) Inventors: Luyan MA, Beijing (CN); Tianhu ZHAO, Beijing (CN); Di WANG, Beijing (CN); Shiwei WANG, Beijing (CN)

(73) Assignee: INSTITUTE OF MICROBIOLOGY, CHINESE ACADEMY OF SCIENCES, Beijing (CN)

(21) Appl. No.: 15/907,221

(22) Filed: Feb. 27, 2018

(30) Foreign Application Priority Data
Sep. 18, 2017 (CN) 201710841101.5
Feb. 22, 2018 (CN) 201810154326.8

(51) Int. Cl. A61K 39/40; A61P 31/04; A61K 31/7105; A61K 31/711

(52) U.S. Cl. CPC A61K 39/40; A61P 31/04; A61K 31/4453; A61K 31/711; A61K 31/7105

(57) ABSTRACT

Methods for removing, reducing, dispersing, disrupting or eradicating biofilms present on a surface may include contacting the biofilm with a composition including an agent that reduces the potency of an aminopeptidase in the biofilm. The agent may reduce expression, secretion or extracellular activity of the aminopeptidase in the biofilm. The biofilm may be formed by P. aeruginosa or P. stutzeri. The composition may enhance the sensitivity of P. aeruginosa to ciprofloxacin. Methods of determining the number of bacterial population in biofilm or a culture containing bacterial aggregate may include dispersing the bacterial aggregate or biofilm by using a composition comprising exogenous PslG. The number of bacteria may be determined based on the dispersed bacteria.

### CROSS-REFERENCE TO RELATED APPLICATION

The present application claims priority of Chinese Application No. 201710841101.5, filed on Sep. 18, 2017 and Chinese Application No. 201810154326.8, field on Feb. 22, 2018, which are incorporated herein by reference in entirety.

### TECHNICAL FIELD

The present invention generally relates to biofilm and bacterial aggregate, specifically relates to methods and compositions for removing, reducing, dispersing, disrupting, or eradicating biofilm by reducing the potency of an aminopeptidase in the biofilm; and methods and compositions for determining the number of bacteria population in the biofilm or a culture containing bacterial aggregate by using PslG to disperse the biofilm or the bacterial aggregate.

### BACKGROUND

“Biofilm” generally refers to communities of microorganism encased by extracellular polymeric substances (EPS), and is prevalent in natural, industrial, and clinical settings. Biofilms enhance survival of the microorganisms, enabling them to adapt to changing conditions collectively instead of as single cells. For example, in medical studies, studies have shown that about 65% of human bacterial infections are related to biofilms, and antibiotics resistance of the microorganisms in the biofilms is hundreds or even thousands of times higher than that in a planktonic state. Biofilm bacteria are also protected from the host immune response, giving rise to chronic infections that are notoriously difficult to eradicate.

In most cases, biofilm bacteria show extreme tolerance to almost all antibiotic classes. One of the most important features of biofilms is self-secreted EPS consisting of mainly polysaccharides, proteins, and extracellular DNA (eDNA), which function as a matrix, or glue, holding biofilm cells together and protecting cells from antibiotics and shearing forces in fluid environments. By forming a matrix-encased multicellular aggregate, cells can also escape engulfment by phagocytic cells in a mammalian host. EPS not only promote bacteria to attach to all kinds of surfaces (for example, a biomedical material or a mucosal surface of a biological organism), but also trap antibiotics or reduce the penetration of antibiotics into the bacterial communities. Therefore, in some cases most drugs can only kill microorganisms on the outer layer of the biofilm, yet leaving the microorganisms inside the biofilm intact, which are the main reason for the generation of antibiotic-resistant mutation. Thus, the biofilm can become a potential source of infections, which may cause refractory infections relating to clinical biofilms.

Pseudomonas aeruginosa (also referred to as “P. aeruginosa”) is an environmental bacterium. It is also an important human pathogen that causes diverse infections in humans. Clinically, P. aeruginosa may cause infections of blood, ears, eyes, skin and soft tissue, bone and joints, endocardium, respiratory system, etc. P. aeruginosa is the common pathogen in hospital-acquired infections, and it is consistently associated with the highest mortality rate (50-80%). It is also the primary pathogen for causing pneumonia, especially in patients suffering from burns or immune deficiency. P. aeruginosa is also the main pathogen that causes the persistent chronic infection in cystic fibrosis patients. The persistence of P. aeruginosa during these infections has been linked to its ability to form biofilms. Due to the intrinsic resistance of P. aeruginosa to antibiotics and its biofilm formation ability, P. aeruginosa infections are difficult to eradicate. In addition, the biofilm formation by P. aeruginosa is also a main cause of metallic corrosion in environment.

Therefore, there is an urgent need for developing preparations (e.g., compositions) and methods for effectively removing, reducing, dispersing, disrupting or eradicating biofilms formed by microorganisms such as P. aeruginosa.

In addition, some clinical isolates, such as rugose small colony variants (RSCV) of P. aeruginosa, may form bacterial aggregates in liquid culture, which are resistant to antibiotics. Biomass of the bacterial aggregates cannot be measured by regular methods, such as a method based on optical density (OD) or colony forming unit (CFU). Many other Pseudomonas species can also form bacterial aggregates at some growth conditions. Thus, there is a need for developing methods for the measurement of bacterial population in biofilm or bacterial aggregates by using the regular methods.

### SUMMARY

The present invention generally relates to methods of removing, reducing, dispersing, disrupting or eradicating biofilm or bacterial aggregate, and methods for determining the number of bacterial population in biofilm or a culture containing bacterial aggregate.

In one aspect of the present invention, a method for removing, reducing, dispersing or disrupting biofilm is provided. The method may include reducing potency of an aminopeptidase in the biofilm. In yet another aspect of the present invention, a composition to remove, reduce, disperse or disrupt biofilm is provided. The composition may reduce the potency of an aminopeptidase in the biofilm. In some embodiments, the method may comprising utilizing the composition herein disclosed.

In some embodiments, to reduce the potency of the aminopeptidase, the method may include contacting the biofilm with a composition that comprises an agent that may reduce expression of the aminopeptidase protein in the biofilm, reduce secretion of the aminopeptidase protein by bacteria in the biofilm, or reduce extracellular activity of the aminopeptidase in the biofilm.

In some embodiments, the agent may comprise an acid agent, an alkaline agent, or a chelating agent.

In some embodiments, the chelating agent may comprise a zinc chelating agent that reduces zinc concentration that maintains the extracellular activity of the aminopeptidase in the biofilm, and the acid or the alkaline disrupts a pH environment that maintains the extracellular activity of the aminopeptidase in the biofilm.

In some embodiments, the agent may comprise an inhibitor that antagonizes the aminopeptidase.

In some embodiments, the inhibitor may be an antibody.

In some embodiments, the inhibitor may be Amastatin or its derivative compounds.

In some embodiments, the agent may reduce expression of the aminopeptidase protein by bacteria in the biofilm. The agent may comprise a small interfering RNA (siRNA), an antisense DNA (asDNA), an antisense RNA (asRNA), or an aptamer.

In some embodiments, the agent may comprise a blocker that reduces the secretion of the aminopeptidase from bacteria in the biofilm by blocking a signal peptide that facilitates the secretion.

In some embodiments, the composition may further comprise an antibiotic.

In some embodiments, the composition may further comprise a pharmaceutically acceptable carrier.

In some embodiments, to reduce the potency of the aminopeptidase in the biofilm, the method may comprise modifying a nucleic acid encoding the aminopeptidase by inserting one or more nucleotides, deleting one or more nucleotides, and/or replacing one or more nucleotides.

In some embodiments, to reduce the potency of the aminopeptidase in the biofilm, the method may comprise reducing secretion of the aminopeptidase by mutating a nucleic acid encoding a signal peptide that facilitates the secretion of the aminopeptidase.

In some embodiments, the biofilm may be formed by P. aeruginosa. The aminopeptidase may have an amino acid sequence of at least 90% identity to SEQ ID NO: 1.

In some embodiments, the composition may enhance the sensitivity of P. aeruginosa to ciprofloxacin.

In some embodiments, the biofilm may be formed by Pseudomonas stutzeri (also referred to as “P. stutzeri”). The aminopeptidase may have an amino acid sequence of at least 90% identity to SEQ ID NO: 3.

In another aspect of the present invention, a method of removing, reducing, dispersing, disrupting or eradicating biofilm is provided. The method comprises contacting the biofilm with a composition that comprises an agent. The agent may reduce expression of the aminopeptidase protein in the biofilm, reduce secretion of the aminopeptidase by bacteria in the biofilm; or reduce extracellular activity of the aminopeptidase in the biofilm. The aminopeptidase may have an amino acid sequence of at least 95% identity to SEQ ID NO: 1 and the biofilm may be formed by P. aeruginosa. Alternatively, the aminopeptidase may have an amino acid sequence of at least 95% identity to SEQ ID NO: 3 and the biofilm may be formed by P. stutzeri.

In some embodiments, the agent may comprise: an acid agent, an alkaline agent, or a chelating agent, which disrupt ion concentration and pH environment that maintain the extracellular activity of the aminopeptidase in the biofilm; an inhibitor that specifically antagonizes the aminopeptidase; an siRNA, an asDNA, an asRNA, or an aptamer, which reduce expression of the aminopeptidase protein by bacteria in the biofilm; or a blocker that reduces the secretion of the aminopeptidase by blocking a signal peptide that facilitates the secretion.

In some embodiments, reducing the potency of the aminopeptidase may cause cell death of bacteria in the biofilm.

In some embodiments, reducing the potency of the aminopeptidase may cause disruption of Psl matrix and thus cause dispersion of the biofilm.

In some embodiments, the composition may further comprise an antibiotic.

In some embodiments, the antibiotic and the agent may have synergistic effects in removing, reducing, dispersing, disrupting or eradicating the biofilm or the bacterial aggregates.

In some embodiments, the antibiotic may be ciprofloxacin or any antibiotics that kill Pseudomonas species.

In yet another aspect of the present invention, a method of determining the number of bacteria population in biofilm or culture containing bacterial aggregate is provided. The method may comprise dispersing the bacteria aggregate or biofilm by using a composition comprising exogenous PslG. The method may also comprise determining the number of bacteria based on the dispersed bacteria.

In some embodiments, the bacterial aggregate or biofilm may be formed by P. aeruginosa or P. stutzeri.

### BRIEF DESCRIPTION OF THE DRAWINGS

The present invention is further described in terms of exemplary embodiments. These exemplary embodiments are described in detail with reference to the drawings, which are used to explain the present invention, and not intended to be limiting, and wherein:

FIG. 1 shows detection of proteins co-purified with EPS prepared from the Psl-overproducing strain WFPA801 and the Psl-negative strain WFPA800;

FIG. 2 shows growth curves of PAO1 and the paaP in-frame deletion mutant ΔpaaP;

FIG. 3A shows comparisons of the initial attachment, the Psl production and aminopeptidase activity of PAO1, ΔpaaP, PaAP Tn5 insertion mutant paap::Tn5, Psl-negative strain ΔPₚₛₗ, Psl and PaAP negative strain ΔPₚₛₗΔpaaP and Psl-inducible strain P_{BAD}-psl on a microtiter dish;

FIG. 3B shows comparisons of the initial attachment, the Psl production, the aminopeptidase activity and the extracellular PaAP of the PAO1/vector, ΔpaaP/vector, ΔpaaP/pPaAP, ΔpaaP/pD308A, ΔpaaP/pPaAPNS;

FIG. 3C shows a comparison of the transcription of lasI::lacZ in PAO1, ΔpaaP, and ΔpaaP/pPaAP;

FIG. 3D shows the relative Psl production of PAO1, ΔpaaP, PAO1/pLasI, and PAO1 supplied with 5 μM C₁₂-HSL;

FIG. 3E shows a comparison of transcription of psl in PAO1, ΔpaaP, and PAO1 supplied with 5 μM C₁₂-HSL detected by the psl::gfp reporter plasmid;

FIG. 4A shows a comparison of the biofilm biomass of PAO1 and ΔpaaP in 6 h of growth in a microtiter dish;

FIG. 4B shows a comparison of the biofilm biomass of PAO1, ΔpaaP, ΔpaaP:PaAP, ΔpaaP::D308A and ΔpaaP:PaAPNS strains after 12 h, 24 h, 36 h, and 48 h of growth in a microtiter dish;

FIG. 4C shows a comparison of Psl production between PAO1 and ΔpaaP strains after 12 h, 24 h, 36 h and 48 h of growth in planktonic culture;

FIG. 4D shows comparisons of aminopeptidase activity and the detection results of extracellular and intracellular PaAP of PAO1, ΔpaaP, ΔpaaP::PaAP, ΔpaaP::D308A and ΔpaaP::PaAPNS;

FIG. 5A is a three-dimensional reconstituted image illustrating the live/dead staining results of the pellicles of PAO1, ΔpaaP and ΔpaaP::PaAP after 24 h, 36 h, and 48 h of growth;

FIG. 5B shows comparisons of pellicle biomass and live and dead bacterial cells in the pellicle biomass of PAO1, ΔpaaP and ΔpaaP::PaAP after 24 h, 36 h, and 48 h of growth;

FIG. 5C shows a comparison of the relative live bacteria in PAO1 and ΔpaaP pellicles;

FIG. 5D shows a comparison of the percentage of dead bacteria in the pellicles shown in FIG. 5A;

FIG. 5E shows the biofilm biomass of PAO1, ΔpaaP and ΔpaaP:PaAP after 24 h, 36 h, and 48 h of growth in a flow-cell system;

FIG. 6A is a three-dimensional reconstituted image illustrating the live/dead staining results of the pellicles of ΔpaaP::D308A and ΔpaaP:PaAP after 24 h, 36 h, and 48 h of growth;

FIG. 6B shows comparisons of biofilm biomass and live and dead bacterial cells in the biofilm biomass of ΔpaaP::D308A and ΔpaaP:PaAP after 24 h, 36 h, and 48 h of growth;

FIG. 6C shows a comparison of the percentage of dead bacteria in the pellicles shown in FIG. 6A;

FIG. 7A shows the top-down view (square) and side view (rectangle) of pellicles of PAO1, ΔpaaP and ΔpaaP::PaAP after 24 h, 36 h, or 48 h of growth, in which biofilm bacteria and the Psl matrix were stained;

FIG. 7B shows a comparison of Psl in pellicles of PAO1, ΔpaaP and ΔpaaP::PaAP after 24 h, 36 h, or 48 h of growth;

FIG. 7C shows a comparison of pellicle biomass of PAO1, ΔpaaP and ΔpaaP::PaAP after 24 h, 36 h, or 48 h of growth;

FIG. 7D shows the volume ratio of Psl to pellicle of PAO1, ΔpaaP and ΔpaaP:PaAP after 24 h, 36 h, or 48 h of growth;

FIG. 8A shows the elution profiles of Psl extracted from 48 h-old PAO1 or ΔpaaP biofilms through Sephadex G-50 column;

FIG. 8B shows the elution profiles of Psl extracted from PAO1 biofilm and PslG treated PAO1 biofilm;

FIG. 8C shows a comparison of extracellular PslG in the PAO1 and ΔpaaP after 48 h of growth;

FIG. 8D shows the biofilm biomass of ΔPₚₛₗ and ΔPₚₛᵢΔpaaP in a microtiter dish over 84 h of growth;

FIG. 8E shows comparisons of pellicle biomass and live/dead bacteria of ΔPₚₛₗ and ΔPₚₛᵢΔpaaP after 36 h of growth and the corresponding optical density at 600 nm (OD₆₀₀) of cultures under the pellicles;

FIG. 8F shows comparisons of biomass of live and dead bacteria in PAO1 and ΔpaaP pellicles after 36 h of growth and their corresponding OD₆₀₀ of cultures under the pellicles;

FIG. 9A shows E test strip results of PAO1, ΔpaaP, and ΔpaaP::PaAP towards ciprofloxacin or tobramycin;

FIG. 9B shows a comparison of relative transcription level of multidrug efflux system genes, mexE, mexF and oprN detected by relative quantitative real-time PCR;

FIG. 10A shows a three-dimensional reconstituted image illustrating the live/dead staining results of the pellicles of P. stutzeri strain A1501 and aminopeptidase deletion mutant strain A1501 (AP::Gm) after 60 h of growth;

FIG. 10B shows a comparison of biofilm biomass and live and dead bacterial cells in the biofilm biomass of A1501 and A1501 (AP::Gm) shown in FIG. 10A;

FIG. 11A shows the biofilms of PAO1 and ΔpaaP grown at 30° C. after 24 h in a flow cell system;

FIG. 11B shows the biofilms of PAO1 and ΔpaaP grown at 20° C. after 24 h in a flow cell system;

FIG. 11C shows the biofilm biomass of PAO1, ΔpaaP, and ΔpaaP:PaAP formed in the microtiter dish at 20° C.;

FIG. 11D shows growth curves of PAO1 and ΔpaaP at 20° C.;

FIG. 11E shows PaAP transcription levels in PAO1 at 20° C. and 30° C.;

FIG. 11F shows the PaAP activity of PAO1 grown at 20° C. and 30° C.;

FIG. 11G shows relative Psl production of PAO1 and ΔpaaP in planktonic culture at 20° C.;

FIG. 12 illustrates an inhibiting effect of Amastatin on enzyme activity of PaAP;

FIG. 13A shows the OD values measured for a PAO1-derived rugose small colony variant strain MJK8 after 3 h or 6 h of growth with or without PslG in Jensen's medium;

FIG. 13B shows the OD values measured for the MJK8 after 3 h or 6 h of growth with or without PslG in Luria broth without sodium chloride;

FIG. 13C shows the OD values measured for A1501 after 3 h, 6 h or 30 h of growth with or without PslG in KLG medium;

FIG. 14 shows the live cell counting results by CFU in a control group, a homogenate group and a PslG group for PAO1;

FIG. 15A shows the 24-h growth curves of a control group, a homogenate group and a PslG group obtained based on the concentration of total proteins for MJK8;

FIG. 15B shows the 24-h growth curve for a control group obtained based on the concentration of total proteins and the OD₆₀₀ measurement for MJK8;

FIG. 15C shows the 24-h growth curve for a homogenate group obtained based on the concentration of total proteins and the OD₆₀₀ measurement for MJK8; and

FIG. 15D shows the 24-h growth curve for a PslG group obtained based on the concentration of total proteins and the OD₆₀₀ measurement for MJK8.

### DETAILED DESCRIPTION

The present invention is related to methods for removing, reducing, dispersing, disrupting or eradicating biofilms formed on biotic or abiotic surfaces or bacterial aggregates in liquid. It should be apparent to those skilled in the art that various alterations, improvements and modifications may be made, and are within the spirit and scope of the exemplary embodiments of this invention.

The terminology used herein is for the purpose of describing exemplary embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

In the present disclosure, terms “PaAP protein” and “PaAP” may be used interchangeably, and may refer to an aminopeptidase synthesized by P. aeruginosa or other similar microbial homologous protein. It should be understood that the terms “PaAP protein” and “PaAP” may also include a wild type and a mutant type of the PaAP protein, including a full-length form, a mature form, an active fragment of the PaAP protein and/or a protein derived from the PaAP protein.

In the present disclosure, terms “PslG protein” and “PslG” may be used interchangeably, and may be derived from a protein with unknown functions synthesized by P. aeruginosa or other similar microbial homologous protein. It should be understood that the terms “PslG protein” and “PslG” may also include a wild type and a mutant type of the PslG protein, including a full-length form, a mature form, an active fragment of the PslG protein and/or a protein derived from the PslG protein.

In one aspect of the present invention, a method of removing, reducing, dispersing, disrupting or eradicating biofilm present on a surface is provided, the method including reducing the potency of an aminopeptidase in the biofilm.

In some embodiments, biofilm is a type of biological film formed by microorganisms embedded within a self-secreted extracellular matrix. In certain embodiments, the microorganisms are bacteria. Exemplary bacteria that may form biofilm on a surface may include P. aeruginosa, P. stutzeri, Acinetobacter baumannii, Staphylococcus aureus, Streptococcus pneumonia, Paenibacillus polymyxa, Sinorhizobium meliloti, Bacillus amyloliquefaciens, etc. A surface prone to biofilm formation may include metal, glass, plastic, rock, textiles, wool, a sponge, a human/animal organ or tissue (e.g., a lung, an ear, skin, etc.), or the like, or any combinations thereof.

A self-produced aminopeptidase by the microorganism may be relevant to the formation and/or maintenance of biofilm. For example, the aminopeptidase may benefit the environment adaption of P. aeruginosa at low temperature, such as at 20° C. (as illustrated in Example 11). The aminopeptidase may be important for the biofilm formation at about room temperature or below the room temperature. In some embodiments, the absence of aminopeptidase within biofilms may lead to bacterial cell death during late stages of biofilm development (as illustrated in Examples 6-10). The dead cells may release an enzyme that degrades the EPS, which leads to the dispersion of bacteria from biofilms into effluents. For example, the enzyme that degrades the EPS may include a glycosyl hydrolase referred to as “PslG”. Thus, in some embodiments, biofilm may be removed, reduced, dispersed, disrupted or eradicated by reducing the potency of the aminopeptidase. As used herein, the term “potency” refers to the extracellular catalyzing ability of the aminopeptidase, which may be affected by the concentration of the aminopeptidase in the biofilm and the activity level (e.g., catalyzing units/mol) of the aminopeptidase. The concentration of the aminopeptidase in the biofilm refers to the extracellular concentration of the aminopeptidase, which may be affected by the expression of the aminopeptidase and the secretion of aminopeptidase to an extracellular matrix. The activity of the aminopeptidase may refer to a capacity of catalyzing a reaction of releasing amino acids from the N-terminus of peptide or protein substrates normalized by the concentration of the aminopeptidase. In some embodiments, when the concentration and/or the activity of the aminopeptidase are reduced, the function of the aminopeptidase may be inhibited, leading to an inhibitory effect on the potency of the aminopeptidase in the biofilm. In some embodiments, the reduction of the potency of the aminopeptidase results in the dispersion, removal, reductions, or disruption of the biofilm. In some embodiments, the aminopeptidase is a leucyl aminopeptidase, an alanyl aminopeptidase, a glutamyl aminopeptidase, or an arginyl aminopeptidase, or any combination thereof. In certain embodiments, the aminopeptidase is P. aeruginosa aminopeptidase (PaAP). In some embodiments, the aminopeptidase comprises an amino acid sequence of SEQ ID NO: 1. In some embodiments, the aminopeptidase comprises an amino acid sequence of SEQ ID NO: 3. In some embodiments, a method for reducing the potency of the aminopeptidase may comprise contacting the biofilm with a composition that comprises an effective agent that reduces expression of the aminopeptidase protein in the biofilm, reduces secretion of the aminopeptidase by the bacteria in the biofilm, or reduces extracellular activity of the aminopeptidase in the biofilm.

In some embodiments, a composition that is a solution, a suspension liquid or emulsion, or any combined forms thereof, containing an effective agent, may be used to immerse, rinse or simply applied to a surface to prevent, remove, reduce, disperse, disrupt or eradicate biofilm that has been developed or will develop on the surface. Solid forms of the composition containing the effective agent may be applied, such as cream, power, particles or nanoparticles, etc. The composition may also be applied to the biofilm directly or indirectly. For example, the composition may be administered to a patient suffering from a biofilm related disease by means such as but not limited to the oral route, injection routes (e.g., subcutaneous, intramuscular, intravenous, intrathecal), sublingual and buccal routes, rectal route, vaginal route, ocular route, otic route, and nasal route. In some embodiments, the agent is effective in reducing the potency of the aminopeptidase. In some biofilms, the aminopeptidase is an exopeptidase that is synthesized in a cell, secreted out of the cell, and functions in the extracellular space. In some embodiments, the expression of the aminopeptidase and secretion of the aminopeptidase into an extracellular matrix may affect the concentration of the aminopeptidase in the biofilm. In some embodiments, the agent functions to reduce the potency of the aminopeptidase by reducing expression and/or secretion of the aminopeptidase, which may affect the extracellular concentration of the aminopeptidase in the biofilm. In some embodiments, the agent reduces the potency of the aminopeptidase by reducing the extracellular activity of the aminopeptidase.

In some embodiments, the agent included in the composition for reducing the extracellular activity of the aminopeptidase may include an acid agent, an alkaline agent, or a chelating agent.

Extracellular activity of the aminopeptidase may be maintained by secondary, tertiary and quaternary structure of the protein. The secondary structure may be regular conformation of a backbone of a peptide, including α-helix, β-pleated sheet, random coil, etc. The tertiary structure may be a complete spatial conformation of a peptide. The quaternary may be a more complex spatial conformation by a plurality of peptides of a protein via non-covalent bonds. Changes in a structure of the aminopeptidase may lead to protein denaturation and/or failure to perform relevant biological functions by the aminopeptidase.

Decreasing or increasing the pH beyond a certain level may lead to protein denaturation. A chelating agent may bind certain functional groups of the aminopeptidase or some metal ions (e.g., Zn²⁺, Mg²⁺) that are required for the proper biological functions of the aminopeptidase. There are other methods to reduce the extracellular activity of aminopeptidase, such as raising the temperature, exposure to ultraviolet rays, x-rays, ultrasound, etc. Some methods are more suitable if they bring less extra damage to the surface that the biofilm attaches to or require extra equipment or facilities. In some embodiments, for removing, reducing, dispersing or disrupting biofilm, the biofilm is contacted with a composition including an acid agent, an alkaline agent or a chelating agent to reduce the potency of the aminopeptidase.

In some embodiments, the chelating agent may comprise a zinc chelating agent that reduces zinc concentration that maintains the extracellular activity of the aminopeptidase in the biofilm, and the acid agent or the alkaline agent may disrupt a pH environment that maintains the extracellular activity of the aminopeptidase in the biofilm.

Exemplary zinc chelating agents may include but not be limited to tetraethylene pentamine, 1,10-phenanthroline. 4,7- and 1,7-phenanthroline, Ethylenediaminetetraacetic acid (EDTA), N,N,N′,N′-tetrakis-(2-pyridylmethyl)ethylenediamine (TPEN), diethylenetriaminepentaacetic acid (DTPA), N,N-diethyldithiocarbamate (DEDTC), or the like, or a combination thereof. The acid agents may include but not be limited to a hydrochloric acid, a sulfuric acid, a sulphurous acid, a nitric acid, a chloric acid, an acetic acid, a phosphoric acid, an oxalic acid, or the like, or any combination thereof. Exemplary alkaline agents may include but not be limited to a sodium hydroxide, a potassium hydroxide, an ammonium hydroxide, a calcium hydroxide, a barium hydroxide, or the like, or a combination thereof.

In some embodiments, for dispersing, removing, reducing or disrupting biofilm on a surface, the biofilm is contacted with a composition that comprises an inhibitor that antagonizes the aminopeptidase. In certain embodiments, the inhibitor may specifically antagonize the aminopeptidase.

In some embodiments, the aminopeptidase may include one or more aminopeptidases, for example, a leucyl aminopeptidase, an alanyl aminopeptidase, a glutamyl aminopeptidase, an arginyl aminopeptidase, etc. In some embodiments, the inhibitor may be a small molecule antagonist that nonspecifically inhibit the activity of the one or more aminopeptidases from the group of aminopeptidases. Such inhibitors may include but not be limited to (2S,3R)-3-Amino-2-hydroxy-5-methylhexanoyl-Val-Val-Asp (Amastatin), N-[(2S,3R)-3-Amino-2-hydroxy-4-phenylbutyryl]-L-leucine hydrochloride (Bestatin hydrochloride), 3-[[1-[(2-(Hydroxymethyl)-1-pyrrolidinyl)carbonyl]-2-methylpropyl]carbamoyl]octanohydroxamic acid (Actinonin), (2R,5S)-5-Amino-8-guanido-4-oxo-2-phenylmethyloctanoic Acid (Arphamenine A), 3,11-Dihydroxy-2,4,6,8,10,12-hexamethyl-9-oxo-6-tetradecenoic acid 1,3-lactone (Ebelactone A), [(2R,3R)-3-Amino-2-hydroxy-5-methylhexanoyl]-Val-Val-Asp (Epiamastatin), cyclopentyl (2S)-2-[[(2R)-2-[(1S)-1-hydroxy-2-(hydroxyamino)-2-oxoethyl]-4-methylpentanoyl]amino]-2-phenylacetate (Tosedostat), or the like, or a combination thereof. In some embodiments, the inhibitor may be a small molecule antagonist that specifically antagonizes a type of aminopeptidase. Such inhibitors may include N-[(2S,3R)-3-Amino-2-hydroxy-4-phenylbutanoyl]-L-prolyl-L-prolyl-L-alaninamide (Apstatin), 5-amino-8-guanidino-2-(4-hydroxyphenylmethyl)-4-oxooctanoic acid (Arphamenine B), etc.

In some embodiments, the inhibitor includes an antibody to the aminopeptidase. An antibody may be a Y-shaped protein that specifically recognizes a target and binds the target. In some embodiments, the antibody antagonizes the activity of the target. In some embodiments, the antibody may include but not be limited to a non-human derived antibody (e.g., derived from a rat, a camel), a human derived antibody, a humanized antibody, a chimeric antibody, a Fab fragment, an scFV fragment, a disulfide-bond Fv (sdFv) fragment, an anti-idiotype (anti-Id) antibody, an epitope-binding fragment of the antibodies thereof, etc. In some embodiments, the antibody for the aminopeptidase may be produced using bioengineering technologies. In some embodiments, the antibody may be a monoclonal antibody with relatively high specificity, or a polyclonal antibody with relatively low specificity.

In some embodiments, for dispersing, removing, reducing or disrupting biofilm on a surface, the biofilm is contacted with a composition that comprises an agent that reduces the expression of the aminopeptidase. In certain embodiments, the agent reduces the expression of the aminopeptidase protein by cells in the biofilm, directly or indirectly. For example, the agent may be a transcription inhibitor that specifically or non-specifically targets the transcription of the aminopeptidase DNA to RNA; the agent may be a translation inhibitor that specifically or non-specifically targets the translation of the aminopeptidase RNA to protein.

In some embodiments, the agent that reduces the expression of the aminopeptidase protein by bacteria in the biofilm may comprise a small interfering RNA (siRNA), an antisense DNA (asDNA), an antisense RNA (asRNA), or an aptamer. An siRNA is a double-stranded RNA molecule of 20-25 base pairs in length, with one strand complementary to the target messenger RNA (mRNA). An asDNA and an asRNA are single-stranded and may be complementary to the target mRNA. In some embodiments, the asDNA, the asRNA and/or the complementary strand of the siRNA may be used to bind to an mRNA that encodes the aminopeptidase in the biofilm through a complementary base pairing effect, where the mRNA may be degraded by a ribozyme. An aptamer may be an oligonucleotide or peptide that binds to a specific target molecule. In some embodiments, the aptamer may bind to the mRNA that encodes the aminopeptidase in the biofilm and inhibit the expression of the aminopeptidase.

In some embodiments, for dispersing, removing, reducing or disrupting biofilm on a surface, the biofilm is contacted with a composition that comprises a blocker that reduces the secretion of the aminopeptidase in the biofilm. In some embodiments, the blocker reduces the secretion of the aminopeptidase by blocking a signal peptide that facilitates the secretion. A signal peptide is a short peptide that plays an important role in protein, in which the signal peptide may prompt a cell to translocate the protein. In some embodiments, the agent comprises a blocker that reduces the expression of a signal peptide for the aminopeptidase, thus inhibiting the translocation as well as the secretion of the aminopeptidase and reducing the potency of the aminopeptidase. Similar methods of reducing the expression of the aminopeptidase may be applied to reducing the expression of the signal peptide for the aminopeptidase. Specifically, in some embodiments, the agent may comprise an siRNA, an asDNA, an asRNA, or an aptamer that may bind to an mRNA that encodes the signal peptide for the aminopeptidase may be constructed as a blocker. In some embodiments, the agent may comprise a small molecule that blocks the function of the signal peptide. The blocker may function to reduce the expression of the signal peptide that facilitates the secretion of the aminopeptidase. In some embodiments, the signal peptide comprises an amino acid sequence of SEQ ID NO: 2. In some embodiments, the signal peptide comprises an amino acid sequence of SEQ ID NO: 4. In some embodiments, the signal peptide comprises an amino acid sequence having at least 70%, 75%, 80%, 85%, 90%, 95%, or 99% sequence identity to SEQ ID NO: 2. In some embodiments, the signal peptide comprises an amino acid sequence having at least 70%, 75%, 80%, 85%, 90%, 95%, or 99% sequence identity to SEQ ID NO: 4.

In some embodiments, the composition may further comprise an antibiotic. As described previously, biofilm may provide structural support and protection for bacteria embedded in the biofilm and contribute to the bacteria's resistance to antibiotics and/or unfavorable environment. In some embodiments, the bacteria may be exposed after the biofilm is disrupted by using the agent that reduces the potency of the aminopeptidase, thus making the bacteria more susceptible to the effects of the antibiotic. In some embodiments, the agent may enhance the effects of the antibiotic in the treatment of the bacteria infection in a patient. In some embodiments, the agent that reduces the potency of the aminopeptidase and the antibiotic have synergistic effects on killing bacteria in the biofilm.

In some embodiments, the antibiotic may include amoxicillin, doxycycline, tetracycline, minocycline, cephalexin, cefuroxime, ceftriaxone, ciprofloxacin, moxifloxacin, clindamycin, lincomycin, clarithromycin, azithromycin, sulfasalazine, sulfisoxazole, dalbavancin, oritavancin, gentamicin, tobramycin, meropenem, doripenem, metronidazole, azithromycin, levofloxacin, or the like, or any combination thereof. In some embodiments, the antibiotic is ciprofloxacin. In some embodiments, the effects of the antibiotic/aminopeptidase-potency-reducing-agent are significantly stronger than the antibiotic alone at the same concentration; in certain embodiments, the effects are at least ten, five, two or 1.5 folds stronger.

In some embodiments, the composition may further comprise a pharmaceutically acceptable carrier.

In some embodiments, a carrier may be used to imbed or load the agent for stabilization, preservation, initiation, targeted delivery, and/or controlled release. The carrier may include particulate cores having a suitable particle size. The carrier may be soluble or insoluble, e.g., a salt (such as sodium chloride or sodium sulfate), sugar (such as sucrose or lactose), sugar alcohol (such as sorbitol), or starch. In some embodiments, the agent may be slowly released over a period of time to maintain a relatively long term of effect. Exemplary carrier for such slow release may be a material with a porous structure, and the agent may be released via diffusion. As another example, the carrier may be used for targeted delivery, in which the release of the agent may be triggered by stimulus, such as changes in pH, application of heat and/or light, etc. In some embodiments, the carrier may also provide protection for the agent against unfavorable conditions. For instance, the agent may be oxidized by oxygen in atmosphere, decomposed when exposed to light, denatured under low or high pH values, degraded by an enzyme, etc. With the protection provided by the carrier, the agent may maintain effective for a longer period of time. These advantages of using the carrier for the agent may be especially desired in medical applications. Pharmaceutically acceptable carrier may be composed of biocompatible, nontoxic and/or biodegradable materials, such as polysaccharides, proteins, synthetic polymers, or the like, or a combination thereof. Various forms of the carrier may be utilized, including liposomes, polymeric micelles, microspheres, nanoparticles, nanofibers, etc.

In some embodiments, to reduce the potency of the aminopeptidase in the biofilm, a nucleic acid encoding the aminopeptidase may be modified.

As described previously in the present disclosure, the activity of the aminopeptidase may be largely dependent on the structure of the aminopeptidase. In some embodiments, modifying a nucleic acid encoding the aminopeptidase inhibits the production of a functional aminopeptidase, wherein the nucleic acid may be a DNA or an RNA. In some embodiments, this may be accomplished by genetic engineering technologies, for example, site-directed mutagenesis, random mutation, etc. For instance, site-directed mutagenesis may include insertion, deletion and/or replacement of one or more nucleotides at a specific site of interest; random mutation may occur at one or more random sites and may be performed via UV radiation, mutagenic chemicals, etc.

In some embodiments, to modify the nucleic acid encoding the aminopeptidase, the method includes: mutating the nucleic acid encoding the aminopeptidase by inserting one or more nucleotides, deleting one or more nucleotides, and/or replacing one or more nucleotides.

In some embodiments, a desired result of mutating the nucleic acid encoding the aminopeptidase may be one or more changes in the amino acid sequence of the aminopeptidase and/or reduced potency of the aminopeptidase. For instance, site-directed mutagenesis targeting amino acids at a catalytic site and amino acids that play an important role in binding with a substrate and/or a cofactor is used to reduce aminopeptidase potency. As another example, mutation directed to amino acids that play an important role in proper folding and/or proper confirmation of the aminopeptidase is used to reduce aminopeptidase potency. In some embodiments, the mutation may also be directed to a noncoding region in the DNA encoding the aminopeptidase, such as a promoter, an intron, etc. In certain embodiments, the modification introduces a change in these regulatory genes, preventing the proper expression of the aminopeptidase, thus reducing the potency of the aminopeptidase in the biofilm. In some embodiments, the modification introduces an insertion and/or a deletion that do not occur in multiples of three nucleotides, causing a frameshift by changing the 3-nucleotide protein reading frame of the genetic sequence encoding the aminopeptidase, thus reducing the potency of the aminopeptidase in the biofilm.

In some embodiments, to reduce the potency of the aminopeptidase in the biofilm, the method includes reducing secretion of the aminopeptidase by mutating a nucleic acid encoding a signal peptide that facilitates the secretion of the aminopeptidase.

The mutation of a nucleic acid encoding a signal peptide that facilitates the secretion of the aminopeptidase may be implemented by inserting one or more nucleotides, deleting one or more nucleotides, and/or replacing one or more nucleotides. For example, site-directed mutagenesis may target at amino acids of the signal peptide that play an important role in the binding of the signal peptide with a corresponding signal-recognition particle (SRP). As a result, the SRP may not be able to recognize the signal peptide and direct the aminopeptidase for translocation. As another example, mutation may also be directed to a noncoding region in the nucleic acid encoding the aminopeptidase, such as a promoter. A change in these regulatory genes may also prevent the proper expression of the signal peptide. In some embodiments, the signal peptide comprises an amino acid sequence of SEQ ID NO: 2. In some embodiments, the signal peptide comprises an amino acid sequence of SEQ ID NO: 4. In some embodiments, the signal peptide comprises an amino acid sequence having at least 70%, 75%, 80%, 85%, 90%, 95%, or 99% sequence identity to SEQ ID NO: 2. In some embodiments, the signal peptide comprises an amino acid sequence having at least 70%, 75%, 80%, 85%, 90%, 95%, or 99% sequence identity to SEQ ID NO: 4.

In some embodiments of the present invention, the composition is used to treat diseases caused by bacteria that form biofilm and/or become more resistant to antibiotics. According to some embodiments of the present invention, a method of treating a biofilm related disease includes: administering an effective amount of a composition that reduces the potency of the aminopeptidase in the biofilm to a patient suffering from the disease. The bacteria in the biofilm and the related diseases may include but not be limited to P. aeruginosa, which may lead to infections of chronic wounds, chronic otitis media, chronic prostatitis, chronic lung infections in cystic fibrosis (CF) patients, etc. As another example, the bacteria may include but not be limited to Streptococcus pneumoniae that may cause community-acquired pneumonia and meningitis in children and the elderly. In some embodiments, biofilm may help Streptococcus pneumoniae become more resistant to oxidative stress and induce competence. Furthermore, biofilm may often form on an inert surface of an implanted biomedical device such as a catheter, a prosthetic cardiac valve, an intrauterine device, etc. The composition of the present invention may effectively remove, reduce, disperse or eradicate biofilm in a mild way without causing extra damage to a biomedical device, a human/animal organ or tissue.

In some embodiments, the method of reducing the potency of an aminopeptidase in the biofilm may be used on biofilm formed by P. aeruginosa. In some embodiments, the composition of the present invention may be used for removing, reducing, dispersing, disrupting or eradicating biofilm formed by P. aeruginosa present on a surface.

As illustrated by Examples 6-8 below, the absence of an aminopeptidase in P. aeruginosa may lead to bacterial cell death in biofilm and biofilm disruption. Therefore, in some embodiments, reducing the potency of the aminopeptidase in P. aeruginosa results in dispersing, removing, reducing, disrupting or eradicating biofilm present on a surface.

In some embodiments of the present invention, the biofilm is formed by P. aeruginos. In some embodiments, the aminopeptidase targeted for removing, reducing, dispersing, disrupting or eradicating the biofilm has a sequence identity of at least 75%, 80%, 85%, 90%, 92%, 95%, 98%, or 99% to SEQ ID NO: 1. In certain embodiments, the aminopeptidase has a sequence identity of at least 90%, 95% or 99% to SEQ ID NO: 1. In some embodiments, the aminopeptidase comprises an amino acid sequence of SEQ ID NO: 1.

In some embodiments, the method of reducing the potency of an aminopeptidase in the biofilm may be used on biofilm formed by P. stutzeri. In some embodiments, the composition of the present invention may be used for removing, reducing, dispersing, disrupting or eradicating biofilm present on a surface.

As illustrated by Example 10 below, the absence of an aminopeptidase in P. stutzeri may lead to biofilm disruption. Therefore, in some embodiments, reducing the potency of the aminopeptidase in P. stutzeri results in removing, reducing, dispersing, disrupting or eradicating biofilm present on a surface.

In some embodiments of the present invention, the biofilm is formed by P. stutzeri. In some embodiments, the aminopeptidase targeted for removing, reducing, dispersing or disrupting the biofilm has a sequence identity of at least at least 75%, 80%, 85%, 90%, 92%, 95%, 98%, or 99% to SEQ ID NO: 3. In certain embodiments, the aminopeptidase has a sequence identity of at least 90%, 95% or 99% to SEQ ID NO: 3. In some embodiments, the aminopeptidase comprises an amino acid sequence of SEQ ID NO: 3.

In another aspect of the present invention, a method of removing, reducing or disrupting biofilm present on a surface is provided. The method includes contacting the biofilm with a composition that comprises an agent that: reduces expression of the aminopeptidase protein in the biofilm; reduces secretion of the aminopeptidase by bacteria in the biofilm; and/or reduces extracellular activity of the aminopeptidase in the biofilm. In some embodiments, the aminopeptidase has an amino acid sequence of at least 95% identity to SEQ ID NO: 1 and the biofilm is formed by P. aeruginosa. In some embodiments, the aminopeptidase has an amino acid sequence of at least 95% identity to SEQ ID NO: 3 and the biofilm is formed by P. stutzeri.

The composition of the present invention may be applied to the biofilm at any time. In some embodiments, the composition of the present invention may be applied to the biofilm after the biofilm has been formed for more than 0, 6, 12, 18, 24, 30, 36, 42, 48, 54, 60, 66, or 72 hours. In some embodiments, the composition of the present invention may be applied to the biofilm after the biofilm has been formed for more than 12, 24, 36 or 48 hours. In some embodiments, the composition of the present invention may be applied to the biofilm after the biofilm has been formed for more than 24 or 36 hours. In some embodiments, the composition of the present invention may be applied every 6, 12, 18, 24, 36 or 48 hours.

In some embodiments, the agent may include: an acid agent, an alkaline agent, or a chelating agent, which disrupts ion concentration and pH environment that maintain the extracellular activity of the aminopeptidase in the biofilm. In some embodiments, the agent may include an inhibitor that specifically antagonizes the aminopeptidase. In some embodiments, the agent may include an siRNA, an asDNA, an asRNA, or an aptamer, which reduces expression of the aminopeptidase protein by bacteria in the biofilm, a blocker that reduces the secretion of the aminopeptidase by blocking a signal peptide that facilitates the secretion.

In some embodiments, the method of the present invention includes contacting a biofilm with a composition to cause bacterial cell death in the biofilm, wherein the composition comprises an agent that reduces the potency of aminopeptidase in the biofilm. As indicated in Example 7, in some embodiments, reducing the potency of the aminopeptidase causes cell death of bacteria in the biofilm. In some embodiments, the method of the present invention relates to treating a disease by causing the cell death of bacteria associated with the disease.

In some embodiments, reducing the potency of the aminopeptidase causes disruption of Psl matrix and thus causes dispersion of the biofilm. Psl is a matrix exopolysaccharide and plays an important role in biofilm (e.g., P. aeruginosa biofilm) formation. In some embodiments, Psl forms a fiber-like web that enmeshes bacterial communities and covers biofilm. In addition, this exopolysaccharide also plays an important role for the biofilm formation of mucoid strains, and can function as a signal to stimulate biofilm formation. As illustrated in Examples 6-8, dead bacteria cells caused by reducing the potency of the aminopeptidase may release PslG, an enzyme that degrades Psl. In some embodiments, the effects of an agent to reduce the potency of the aminopeptidase and disperse the biofilm is mediated by Psl. In some embodiments, reducing the potency of the aminopeptidase causes bacterial cell death and the release of PslG, thus disrupting the biofilm.

In yet another aspect of the present invention, a method of removing, reducing or disrupting biofilm present on a surface is provided. In some embodiments, the method includes contacting the biofilm with a composition that comprises an antibiotic and an agent that: reduces expression of the aminopeptidase protein in the biofilm; reduces secretion of the aminopeptidase by bacteria in the biofilm; and/or reduces the extracellular activity of the aminopeptidase in the biofilm In some embodiments, the aminopeptidase has an amino acid sequence having at least 95% identity to SEQ ID NO: 1 and the biofilm is formed by P. aeruginosa. In some embodiments, the aminopeptidase has an amino acid sequence having at least 95% identity to SEQ ID NO: 3 and the biofilm is formed by P. stutzeri.

In some embodiments, the antibiotic and the agent may have synergistic effects in removing, reducing or disrupting the biofilm.

In some embodiments, disrupting the biofilm may enhance the sensitivity of the bacteria to the antibiotics. Such bacteria may include bacteria that may form biofilm on a solid surface and planktonic bacteria that may form biofilm or bacteria aggregates in liquid environments. In some embodiments, the bacteria may include wild type P. aeruginosa and some clinical isolates of P. aeruginosa, such as the RSCV strains of P. aeruginosa.

As illustrated in Examples 6, 7 and 10 below, reducing the potency of the aminopeptidase in the biofilm formed by P. aeruginosa or P. stutzeri may lead to biofilm disruption and bacteria death. Once the biofilm is disrupted, the bacteria may lose the protection and structural support provided by the biofilm. Therefore, the antibiotic may effectively kill the exposed live bacteria. As a result, the sensitivity of the bacteria to antibiotics is enhanced. In some embodiments, dead bacteria cells may release more PslG, an enzyme that degrades Psl matrix, and thus facilitating the disruption of the remaining biofilm. Therefore, the antibiotic and the agent that reduces the potency of the aminopeptidase may have synergistic effects in killing microorganisms (e.g., bacteria) that form biofilm or aggregates. In some embodiments, the synergistic effect of the antibiotic and the agent may be especially desirable in medical applications, for example, treating infections associated with biofilm in human/animal body tissue or organs, and/or implanted medical devices such as a catheter, a prosthetic cardiac valve an intrauterine device, etc.

In some embodiments, the antibiotic may be ciprofloxacin, or any antibiotics that kill Pseudomonas species.

Reducing the potency of the aminopeptidase may enhance the sensitivity of P. aeruginosa to antibiotics such as ciprofloxacin. In some embodiments, the increased sensitivity is due to decreased expression of a multidrug efflux operon as indicated in Example 9. In some embodiments, ciprofloxacin may be an effective antibiotic to be used in combination with the agent to remove, reduce or disrupt biofilm presenting on a surface.

In yet another aspect of the present invention, a method of determining a number of bacterial communities in biofilm or bacterial aggregates is provided. In some embodiments, the method comprises preventing or dispersing bacteria aggregates or biofilm by using a composition including an agent that contains PslG and determining the number of bacteria based on the dispersed bacteria.

In some embodiments, the composition including the agent that reduces the potency of the aminopeptidase (e.g., PaAP) may be used to contact the bacteria aggregate to enhance their sensitivity to antibiotics. In some embodiments, the composition may be added to a growth medium of the bacteria.

In some embodiments, PslG may be used to contact the bacteria aggregate to disperse the aggregate for enhancing the sensitivity of bacterial aggregate to antibiotics. In some embodiments, the PslG may be added to a growth medium of the bacteria. For example, in some embodiments, a growth state for the bacteria needs to be monitored or a growth curve for the bacteria needs to be measured. Bacteria from the growth medium containing the PslG may disperse or prevent bacterial aggregation. Thus, bacterial population may be eradicated by antibiotics.

Exemplary methods for determining the number of bacteria may include but not be limited to a method based on a counting chamber, a method based on plating and colony forming units (CFUs), a method based on a coulter counter, a method based on flow cytometry, a method based on image analysis, a method based on spectrophotometry, a method based on impedance microbiology, or the like, or a combination thereof.

In some embodiments, in the method of dispersing bacteria aggregate or biofilm by using a composition including PslG protein that degrades Psl matrix and determining the number of bacteria based on the dispersed bacteria.

In some embodiments, the composition may include PslG and other enzymes that can degrade the EPS. The formation of the biofilm may be associated with different polysaccharides, such as Pel, alginate, etc. Thus, apart from PslG, the composition may also include proteins that can degrade Pel, alginate, or other types of polysaccharides.

In some embodiments, the encoding sequence of the PslG protein may be a nucleotide sequence of SEQ ID NO: 5. In some embodiments, the PslG protein may have an amino acid sequence of SEQ ID NO: 6.

In some embodiments, the PslG protein may be selected from: (i) a protein having an amino acid sequence of SEQ ID NO: 6; (ii) a protein derived from (i), with one or more amino acid residues being substituted in, deleted from, and/or added into the amino acid sequence of SEQ ID NO: 6, wherein the derived protein may be capable of inhibiting formation of biofilm or degrading biofilm; and (iii) a protein that has at least 95% or at least 98% amino acid sequence identity to SEQ ID NO: 6, wherein the protein may be capable of inhibiting formation of biofilm or degrading biofilm. In some embodiments, the ability of the protein of inhibiting formation of biofilm or degrading biofilm may be improved by modifying certain amino acids in the sequence of SEQ ID NO: 6.

For example, the capacity of binding with the substrate may be enhanced. As another example, the structural stability and the catalytic activity of the protein under unfavorable conditions (e.g., low/high pH, low/high temperature) may be improved.

In some embodiments, the encoding sequence of the PslG protein may be selected from: (a) a nucleic acid that encodes a protein having the amino acid sequence of SEQ ID NO: 6; (b) a nucleic acid having a nucleotide sequence of SEQ ID NO: 5; (c) a nucleic acid having at least 95% or at least 98% nucleotide sequence identity to SEQ ID NO: 5; (d) a nucleic acid having a nucleotide sequence which results from: 1-60, 1-30, or 1-10 nucleotides being truncated from or added to the 5 end or 3 end of the nucleotide sequence of SEQ ID NO: 5; (e) a nucleic acid having a nucleotide sequence complementary to the nucleotide sequence in any one of (a) to (d).

In some embodiments, the composition may include the PslG protein, an active fragment of the PslG protein, an appropriate carrier, or other agents. The active fragment may be a fragment of the PslG protein, wherein the fragment is able to prevent or disperse biofilms or bacteria aggregates. The carrier may be a solvent or a solution for dissolving the PslG protein, the active fragment of the PslG protein or other agents, such as water, a saline solution, etc. Other agents may include a buffer agent, a pH modifier, an agent that increases the capacity of preventing or dispersing the biofilm or the bacteria aggregate of the PslG protein.

In some embodiments, the method of dispersing the biofilm or the bacteria aggregate may also include one or more of magnetic agitation, mechanical agitation, vortex, ultrasound treatment, tissue homogenate, or the like, or any combination thereof. The PslG treatment may be used in combination with one or more of the methods mentioned above.

In some embodiments, when the composition is used to prevent or disperse the biofilm or the bacteria aggregate, an effective concentration of the PslG protein may be 0.1 nM-10 μM. In some embodiments, the effective concentration of the PslG protein may be 0.1-500 nM. Alternatively, the effective concentration of the PslG protein may be 5-100 nM. For example, the effective concentration of the PslG protein may be 25 nM, 50 nM, 100 nM, etc.

In some embodiments, when the composition is used to prevent or disperse the biofilm or the bacteria aggregate, the temperature may be 5-75° C. In some embodiments, the temperature may be 10-60° C. Alternatively, the temperature may be 15-50° C. For example, the temperature may be 30° C.

In some embodiments, the composition including PslG may be used to contact the bacteria aggregate or biofilm before determining the number of the bacteria. In some embodiments, the composition including PslG may be added to a growth medium of the bacteria. For example, in some embodiments, a growth state for the bacteria needs to be monitored or a growth curve for the bacteria needs to be measured. Bacteria from the growth medium containing PslG may disperse bacterial aggregates or prevent the forming of aggregates. Thus the determination of the number of bacteria during growth may be more precise and more convenient.

Exemplary methods for determining the number of bacteria may include but not be limited to a method based on a counting chamber, a method based on plating and colony forming units (CFUs), a method based on a coulter counter, a method based on flow cytometry, a method based on image analysis, a method based on spectrophotometry, a method based on impedance microbiology, or the like, or a combination thereof.

In some embodiments, in the method of dispersing bacteria aggregate or biofilm by using a composition including PslG and determining the number of bacteria based on the dispersed bacteria, the bacteria aggregate or biofilm may be formed by P. aeruginosa or P. stutzeri (as illustrated in Example 13).

The present invention is further described by the following examples which should not be construed as limiting the scope of the present invention.

### EXAMPLES

### Materials

Chemicals used as buffers and reagents were commercial products of at least reagent grade. Luria broth (LB) solid medium was prepared using 5 g of yeast extract, 10 g of tryptone, 10 g of NaCl, 20 g of agarose and 1000 ml of distilled water. LB liquid medium was prepared using 5 g of yeast extract, 10 g of tryptone, 10 g of NaCl and 1000 ml of distilled water. LBNS (Luria broth without sodium chloride) solid medium was prepared using 5 g of yeast extract, 10 g of tryptone, 20 g of agarose and 1000 ml of distilled water.

Lombard-Dowell (LD) solid medium was prepared using 5 g of yeast extract, 10 g of tryptone, 2.5 g of NaCl, 20 g of agarose and 1000 ml of distilled water. LD liquid medium was prepared using 5 g of yeast extract, 10 g of tryptone, 2.5 g of NaCl and 1000 ml of distilled water. Jensen's liquid medium was prepared using 5 g of NaCl, 2.51 g of K²HPO₄, 15.56 g of monosodium I-glutamate, 2.81 g of valine, 1.32 g of phenylalanine, 13.87 g of glucose, 0.165 g of MgSO₄.7H₂O, 0.105 mg of CaCl₂.2H₂O, 5.5 μg of FeSO₄.7H₂O, 12 μg of ZnSO₄.7H₂O and 1000 ml of distilled water.

1 L of KLN⁻ liquid medium (pH 6.8) included 1.67 g of K₂HPO₄, 0.87 g of KH₂PO₄, 0.29 g of MgSO₄, 0.48 g of NaCl, 3.78 g of sodium lactate, 0.07 g of CaCl₂, 10 mg of FeCl₃, 5 mg of MaMoO₄, 0.25 mg of MnSO₄.H₂O, 0.072 mg of ZnSO₄.7H₂O, 0.0125 mg of CuSO₄.5H₂O, 0.014 mg of CoSO₄.7H₂O, 0.003 mg of H₃BO₄ and distilled water. 1 L of KLG⁺ medium included 1.67 g of K₂HPO₄, 0.87 g of KH₂PO₄, 0.29 g of MgSO₄, 0.48 g of NaCl, 3.78 g of sodium lactate, 3.38 g of sodium glutamate, 0.07 g of CaCl₂, 10 mg of FeCl₃, 5 mg of MaMoO₄, 0.25 mg of MnSO₄.H₂O, 0.072 mg of ZnSO₄.7H₂O, 0.0125 mg of CuSO₄.5H₂O, 0.014 mg of CoSO₄.7H₂O, 0.003 mg of H₃BO₄ and distilled water (See e.g., Biofilm formation enables free-living nitrogen-fixing rhizobactena to fix nitrogen under aerobic conditions. The ISME journal. 2017; 11(7):1602-13.).

### Bacteria Strains and Vectors

PAO1 is a wild type of Pseudomonas aeruginosa (also referred to as “P. aeruginosa”) that expresses PaAP (See, e.g., Genetic recombination in Pseudomonas aeruginosa. J Gen Microbiol, 1955. 13: 572-581).

ΔpaaP is an in-frame deletion of paap strain of P. aeruginosa. ΔpaaP was prepared by in-frame deletion of gene locus PA2939 (designated as paaP in the present invention) of P. aeruginosa (See e.g., Precision-engineering the Pseudomonas aeruginosa genome with two-step allelic exchange. Nat Protoc, 2015. 10: p. 1820-4); the plasmid for knocking-out paaP was pEX18AP (See e.g., A broad-host-range Flp-FRT recombination system for site-specific excision of chromosomally-located DNA sequences: application for isolation of unmarked Pseudomonas aeruginosa mutants. Gene, 1998. 212(1): p. 77-86); primer sequences included SEQ ID NO: 7 (Up-F: AGAATT GAGGTTCTCG TCTTCAGG), SEQ ID NO: 8 (Up-R: GATCTGGCTGGCGCTCTTCTGCATGTGAGGCGATGATC GATAAGC), SEQ ID NO: 9 (Down-F: GCTTATCGATCATCGCCTCACATGCAGAAGAGCGCCA GCCAGATC), and SEQ ID NO: 10 (Down-R: GTCAAGCTTCTGCTGGTCTGTAGCGAGGAC).

paaP::Tn5 is a paap Tn5 insertion mutant of P. aeruginosa (See e.g., Comprehensive transposon mutant library of Pseudomonas aeruginosa. Proc Natl Acad Sci USA, 2003. 100: 14339-14344.)

WFPA800 is a Psl-negative strain of P. aeruginosa. WFPA800 was prepared by deleting psl operon promoter (See e.g., Analysis of Pseudomonas aeruginosa conditional psl variants reveals roles for the Psl polysaccharide in adhesion and maintaining biofilm structure post attachment. J Bacteriol, 2006. 188: 8213-8221.).

WFPA801 is a Psl-overproducing strain of P. aeruginosa, P_{BAD}-psl (See e.g., Analysis of Pseudomonas aeruginosa conditional psl variants reveals roles for the Psl polysaccharide in adhesion and maintaining biofilm structure postattachment. J Bacteriol, 2006. 188: 8213-8221.).

ΔpaaP::D308A is a mutant strain of P. aeruginosa that expresses inactivated PaAP. ΔpaaP::D308A was prepared by replacing the paaP by the active site mutated paaP (D308A) in P. aeruginosa (See e.g., Precision-engineering the Pseudomonas aeruginosa genome with two-step allelic exchange. Nat Protoc, 2015. 10: 1820-1841.). The plasmid used to prepare the ΔpaaP:D308A was pEX18Gm. The sequences of the primers were SEQ ID NO: 11 (Up-F2: GGAATTCGATGGTGGTGATGACGATGC), SEQ ID NO: 12 (Up-R2: CTCCGTTCCTTGTGAGGCGATGATCGATAAGC), SEQ ID NO: 13 (D308A-F: CATCGCCTCACAAGGAACGGAGTCTCATGAGC), SEQ ID NO: 14 (D308A-R: CGCTCTTCTGCATCTGCAGCG ACCGCGATTGTG), SEQ ID NO: 15 (Down-F2: GGTCGCTGCAGATGCAGAAGAGCGCCAGCC AGATC), and SEQ ID NO: 16 (Down-R2: CGGGATCCGTGCGACCCTCAACCGTTTC).

ΔpaaP:PaAPNS was prepared by replacing the wild type paaP by the signal peptide truncated paaP in P. aeruginosa (Precision-engineering the Pseudomonas aeruginosa genome with two-step allelic exchange. Nat Protoc, 2015. 10: 1820-1841); the plasmid used to prepare the ΔpaaP:PaAPNS was pEX18Gm (See e.g., A broad-host-range Flp-FRT recombination system for site-specific excision of chromosomally-located DNA sequences: application for isolation of unmarked Pseudomonas aeruginosa mutants. Gene, 1998. 212(1): p. 77-86). The sequences of the primers were SEQ ID NO: 17 (Up-F3: GGAATTCGATGGTGGTGATGACGATGC), SEQ ID NO: 18 (Up-R3: CGAAGGTGCCATGAGAC TCCGTTCCTTGTGAG), SEQ ID NO: 19 (PaAPNS-F: GAACGGAGTCTCATGGCACCTTCGGA AGCGC), SEQ ID NO: 20 (PaAPNS-R: CGCTCTTCTGCATCTGCAGCGACCGCGATTGTG), SEQ ID NO: 21 (Down-F3: GGTCGCTGCAGATGCAGAAGAGCGCCAGCCAGATC), and SEQ ID NO: 22 (Down-R3: CGGGATCCGTGCGACCCTCAACCGTTTC).

ΔpaaP:PaAP is an aminopeptidase complemented strain, in which ΔpaaP was complemented by paaP with its own promoter integrated at attB/P site in P. aeruginosa (Integration-proficient plasmids for Pseudomonas aeruginosa: site-specific integration and use for engineering of reporter and expression strains. Plasmid, 2000, 43(1): 59-72.). The plasmid used was mini-CTX-lacZ (Integration-proficient plasmids for Pseudomonas aeruginosa: site-specific integration and use for engineering of reporter and expression strains. Plasmid, 2000, 43(1): 59-72.). The sequences of primers were SEQ ID NO: 23 (PaAP-F2: GGAATTCCGGGAAGAATTTGGTGATG), SEQ ID NO: 24 (PaAP-R2: CGGGATCCTTACTTGATGAAGTCGTGAC).

MJK8 is a PAO1-derived rugose small colony variant (RSCV) strain (See e.g., Pseudomonas aeruginosa rugose small colony variants have adaptions that likely promote persistence in the cystic fibrosis lung. J. Bacteriol. 191:3492-3503).

A1501 is a wild type of Pseudomonas stutzeri (also referred to as P. stutzeri) that expresses aminopeptidases (See, e.g., Nitrogen fixation island and rhizosphere competence traits in the genome of root-associated Pseudomonas stutzeri A1501. PNAS, 2008. vol. 105: 7564-7569).

A1501::Gm is an aminopeptidase deletion mutant of P. stutzeri. The plasmid used was pK18mobsacB for aminopeptidase deletion (See e.g., Small mobilizable multi-purpose cloning vectors derived from the Escherichia coli plasmids pK18 and pK19: selection of defined deletions in the chromosome of Corynebacterium glutamicum. Gene, 1994 145: 69-73.). The sequences of the primers were SEQ ID NO: 25 (P1: TATGACATGATTACGAATTCCGTCGAGAAGAT CCATTCGC), SEQ ID NO: 26 (P2: GCATAGTCGGTCTAGAGGCGATCAATGCGAGAGAAG), SEQ ID NO: 27 (P3: ATTGATCGCCTCTAGACCGACTATGCGCAGTTCTTC), SEQ ID NO: 28 (P4: ACGACGGCCAGTGCCAAGCTTCAATCTCGTCCAGTGCAGC).

PHerd20T is an E. coli-P. aeruginosa shuttle plasmid containing arabinose inducible P_{BAD} promoter and Ap^{r} (See e.g., P_{BAD}-based shuttle vectors for functional analysis of toxic and highly regulated genes in Pseudomonas and Burkholderia spp. and other bacteria. Appl Environ Microbiol 74: 7422-7426.)

PAO1/vector is PAO1 containing an empty vector pHerd20T that does not express PaAP.

ΔpaaP/vector is ΔpaaP containing an empty vector pHerd20T that does not express PaAP.

ΔpaaP/pPaAP is ΔpaaP containing a recombinant plasmid that expresses PaAP. The recombinant plasmid pPaAP was prepared by inserting intact paap into pHerd20T (See e.g., P_{BAD}-based shuttle vectors for functional analysis of toxic and highly regulated genes in Pseudomonas and Burkholderia spp. and other bacteria. Appl Environ Microbiol 74: 7422-7426).

ΔpaaP/pD308A is ΔpaaP containing a recombinant plasmid that expresses inactivated PaAP. The recombinant plasmid pD308A was prepared by inserting PaAP^{D308A} into pHerd20T (See e.g., P_{BAD}-based shuttle vectors for functional analysis of toxic and highly regulated genes in Pseudomonas and Burkholderia spp. and other bacteria. Appl Environ Microbiol 74: 7422-7426).

ΔpaaP/pPaAPNS is ΔpaaP containing a recombinant plasmid that expresses signal peptide truncated PaAP. The recombinant plasmid pPaAPNS was prepared by inserting signal peptide truncated paaP into pHerd20T (See e.g., P_{BAD}-based shuttle vectors for functional analysis of toxic and highly regulated genes in Pseudomonas and Burkholderia spp. and other bacteria. Appl Environ Microbiol 74: 7422-7426).

### Bacterial Growth Conditions and Living Cell Counting by CFUs

Unless otherwise indicated, E. coli strains were cultured at 37° C. in Luria broth (LB), P. aeruginosa strains at 37° C. in LB without sodium chloride (LBNS) or in Jensen's medium, and P. stutzeri strains at 30° C. in LD medium. Antibiotics were added to the appropriate media at the following final concentrations: for E. coli, 100 μg/ml ampicillin and 12.5 μg/ml tetracycline; for P. aeruginosa, 300 μg/ml carbenicillin, 30 μg/ml gentamycin, and 100 μg/ml tetracycline; for P. stutzeri, 30 μg/ml gentamycin. To detect the growth curve, Jensen's medium was used for P. aeruginosa. KLN⁻ medium and KLG⁺ medium was used for P. stutzeri. To detect live bacteria in planktonic cultures, cultures collected at different time points were diluted in PBS, and 10 μl of each dilution was dropped on LBNS plates to calculate the CFUs. To detect live bacteria in the biofilms, biofilms from different time points were washed with PBS and dispersed with 50 nM PslG at 30° C. for 30 minutes, and the number of CFUs was detected accordingly.

### Aminopeptidase Assay

PaAP catalytic activity was determined according to Samovsky's method. To determine the PaAP catalytic activity in the planktonic culture, P. aeruginosa was grown in LBNS for 24 h and then centrifuged to harvest the supernatant. For the PaAP catalytic activity in biofilms, P. aeruginosa biofilms were grown in Jensen's medium for 12 h, 24 h, 36 h and 48 h. Next, the medium and planktonic cells under the biofilms were discarded, and the biofilms were washed with PBS, dispersed with 50 nM PslG at 30° C. for 30 minutes, and centrifuged to harvest the supernatant. Equal volumes of substrate Leu-p-nitroanilide solution (1.2 mM Leu-p-nitroanilide in 0.1 M Tris-HCl, 2 mM CaCl₂, pH 8.3) were added and allowed to react at 50° C. for 15 min. A molar extinction coefficient of 10400 for p-nitroaniline was used to calculate the amount of product released from nitroanilide (NA)-based substrates. PaAP catalytic activity was calculated in nmol of nitroanilide per OD₆₀₀ of the corresponding original culture.

### Biofilms and Image Acquisition

Air-liquid interface biofilms (also referred to as “pellicles” in the present invention) were grown in glass chambers with a glass coverslip at the bottom of each chamber. One milliliter of a 1/100 dilution of P. aeruginosa overnight culture in Jensen's medium or one milliliter of a 1/100 dilution of P. stutzeri overnight culture in KLN⁻ medium was inoculated into the chambers. Biofilms were grown at 30° C. for 24 h, 36 h or 48 h for P. aeruginosa, or 60 h for P. stutzeri. The flow-cell biofilms were grown in a three-channel flow-cell with individual channel dimensions of 1×4×40 mm. The P. aeruginosa mid-log phase culture in Jensen's medium was used for inoculation. After inoculation, the medium flow was stopped for 1 h, resumed at a rate of 1 ml/min, and cultivated at 30° C. The biofilms were stained by using LIVE/DEAD® BacLight™ Bacterial Viability Kits (Molecular Probes, Invitrogen™) to detect dead and live bacteria. For Psl and biofilm double staining, the biofilms were first stained with SYTO9 (5 μM final concentration, Molecular Probes, Invitrogen™) for 15 min. Then, the Psl matrix was stained with lectin TRITC-HHA (Tetramethylrhodamin conjugated Hippeastrum Hybrid Amaryllis) at 100 μg/ml in the dark for 2 h. Fluorescence images were acquired by using a FV1000 confocal laser scanning microscope (CLSM) (Olympus, Japan) with the 63×/1 1.3 objective at 488 nm (for SYTO 9) and 543 nm (for PI or TRITC-HHA). Bitplane Imaris™ 7.2.3 software was used to generate the three-dimensional images and optical Z-sections. CLSM-captured images were subjected to quantitative image analysis using COMSTAT™ software.

### Preparation and Fractionation of Psl Extracted from Biofilms

The Psl extract from biofilm was prepared, fractionated on a Sephadex G-50 column, and irrigated with water at 0.67 ml/min. The total carbohydrate in each fraction (5 ml) was assayed colorimetrically for aldose. Briefly, 200 μl of each fraction was mixed with 100 μl 5% phenol and 500 μl concentrated sulfuric acid, reacted at room temperature for 30 minutes, and measured (OD₄₉₂).

### β-Galactosidase Assay

β-Galactosidase activity was quantitatively assayed.

P. aeruginosa strains were grown in Jensen's medium at 37° C. with shaking at 200 rpm to an OD₆₀₀ of 0.5-0.8. Bacteria from 2 ml culture aliquots were resuspended in 200 ml Z-buffer and frozen/thawed three times to lyse the bacteria. The cell lysates were assayed for both β-galactosidase activities and total proteins using a bicinchoninic acid assay. All β-galactosidase activity units were normalized to the total protein per ml of the aliquots. One unit of β-galactosidase is defined as the amount that hydrolyzes 1 μmol of substrate per minute per mg/ml protein. Immunodot blotting and western blotting

To examine Psl production, exopolysaccharide Psl extracts were collected from overnight cultures equivalent to approximately 10 OD₆₀₀ and examined by immunodot blotting using an anti-Psi antibody. For the effect of C12-HSL on Psl production, 5 μM C12-HSL was added to the medium after inoculation. The software Image Lab™ was used to quantify the immunodot blotting data.

To detect PaAP in EPS, the WFPA801 and WFPA800 strains were cultured on cellophane covered LBNS agar plates with 1% arabinose for 24 h, and then the EPS of WFPA801 and WFPA800 were obtained through treatment with DNase I, RNase, and then proteinase K. 10 μg EPS was dissolved in SDS-PAGE loading buffer and loaded onto a SDS-PAGE gel. The gel was used for western blotting using an anti-PaAP antibody or stained with Coomassie brilliant blue. The protein bands detected in EPS were subjected to mass spectrometry analysis. To detect PaAP in the culture supernatant, an overnight PAO1 culture was diluted 100-fold in Jensen's medium and incubated for 12 h, 24 h, 36 h, and 48 h. The extracellular protein in the culture supernatant was precipitated with 100% trichloroacetic acid (TCA) at a final concentration of 15%. The samples were dissolved in SDS-PAGE loading buffer and detected by western blotting using an anti-PaAP antibody.

To detect extracellular PslG in the biofilm, 48-h biofilms were collected, resuspended in 10 mM Tris-HCl (pH 7.4), dispersed in an ultrasonic bath at 40 KHz for 20 min, and centrifuged at 9000 rpm for 20 min to collect the supernatant. The extracellular proteins in the supernatant were extracted with phenol and then precipitated with acetone and detected by western blotting using an anti-PslG antibody (1:200).

### Relative Quantitative Real-Time PCR

Bacteria were grown to approximately OD_{600˜0.5}, then total RNA was extracted using Trizol® according to manufacturer's specifications (Takara). Genomic DNA was removed by RNase-Free DNase I (NEB, M0303S) for 30 minutes at 37° C., and the RNA was purified using RNA clean Kit (Qiagen™). The total DNase-treated RNA (10 μg) was reversely transcribed to synthesize cDNA using the M-MLV Reverse Transcriptase (Promega™, M1701) with random hexamer primers according to the manufacturer's protocol. 10 ng cDNA was diluted in SYBR Green Mix (Roche®) to do relative quantitative real-time PCR by LightCycler480 (Roche®). Gene expression was normalized by using rpsL as internal control. Relative expression levels of mexE, mexF and oprN were calculated by the relative quantification method (ΔΔC_{T}). All assays were done in triplicates.

### Example 1

### PaAP is Enriched in Psl Matrix

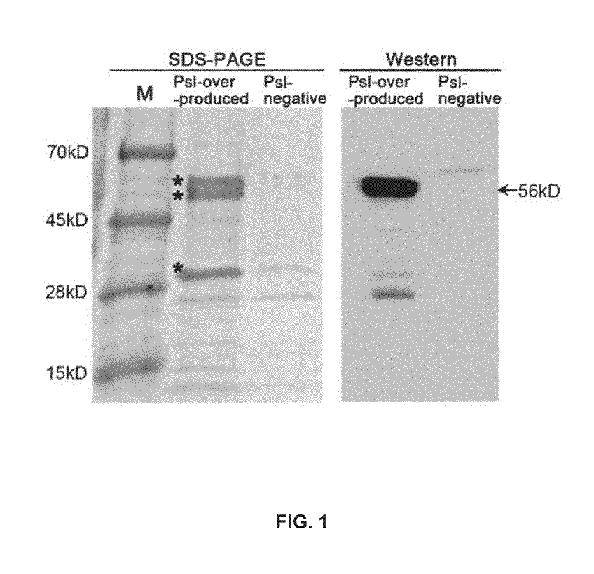

To study the proteins associated with the Psl matrix of P. aeruginosa, EPS was extracted from the P. aeruginosa PAO1-derived Psl-overproducing strain WFPA801 and Psl-negative strain WFPA800 that were cultured on cellophane-covered LBNS agar plates with 1% arabinose. The EPS was analyzed through SDS-PAGE and Coomassie brilliant blue staining. In FIG. 1, the left panel shows Coomassie brilliant blue staining of EPS samples run on an SDS-PAGE gel, and the right panel shows the corresponding western blotting results for the EPS samples detected by using an anti-PaAP antibody. There were only three major protein bands detected in the EPS of WFPA801, wherein two high-molecular-weight bands were≈58-56 KD and the other band was≈28 KD. Mass spectrometry analysis showed that the three bands were all P. aeruginosa aminopeptidase, which was further confirmed by western blotting with an anti-PaAP antibody as illustrated in FIG. 1 (right panel). The two high-molecular-weight bands were also detected in EPS extracted from the wild type strain PAO1 by Coomassie brilliant blue staining and anti-PaAP western blotting. In contrast, little PaAP was detected in the EPS of WFPA800, although there were several detectable protein bands found in the SDS-PAGE gel as illustrated in FIG. 1 (left panel). This example shows that PaAP is enriched in Psl matrix, suggesting the association of PaAP with the Psl matrix of biofilms.

### Example 2

### The Effect of PaAP Deletion on Growth of P. aeruginosa

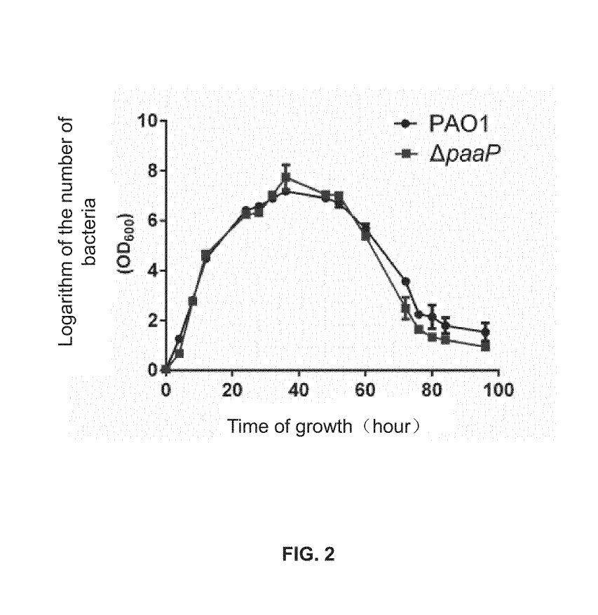

PAO1 and corresponding in-frame deletion mutant, ΔpaaP, were inoculated on LBNS solid medium plates and overnight incubated at 37° C. Then monoclones of the PAO1 and the ΔpaaP, collected from the LBNS solid medium plates, were inoculated in LBNS liquid medium and incubated at 37° C. overnight with shaking at 200 rpm. 1 ml of bacterial solution was inoculated in 100 ml of Jensen's medium and incubated at 37° C. with shaking at 200 rpm. The absorbance at 600 nm of the bacterial solution at different time points was measured using a spectrophotometer and was shown in FIG. 2. FIG. 2 shows that the ΔpaaP mutant has a similar growth rate to that of PAO1 in planktonic cultures. This example suggests that the deletion of PaAP does not have any significant impact on the growth of P. aeruginosa.

### Example 3

### The Effect of PaAP on the Initial Attachment of PAO1

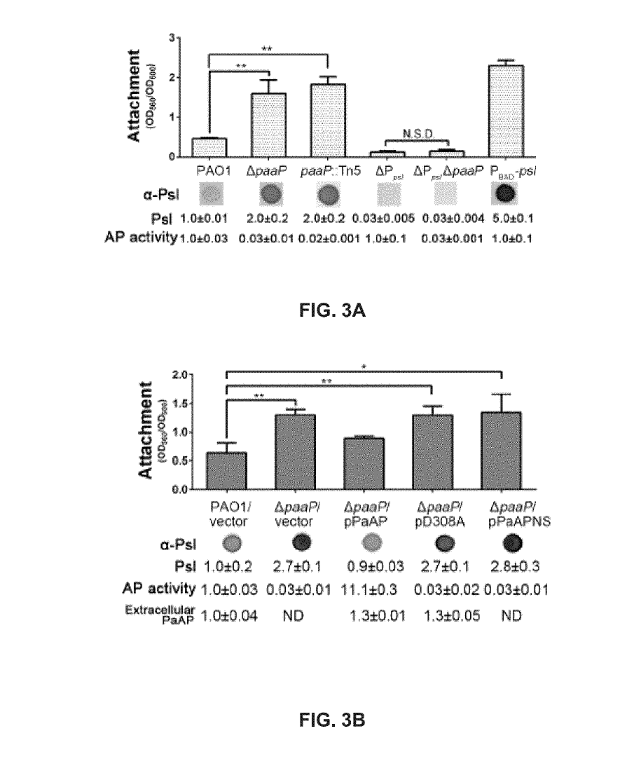

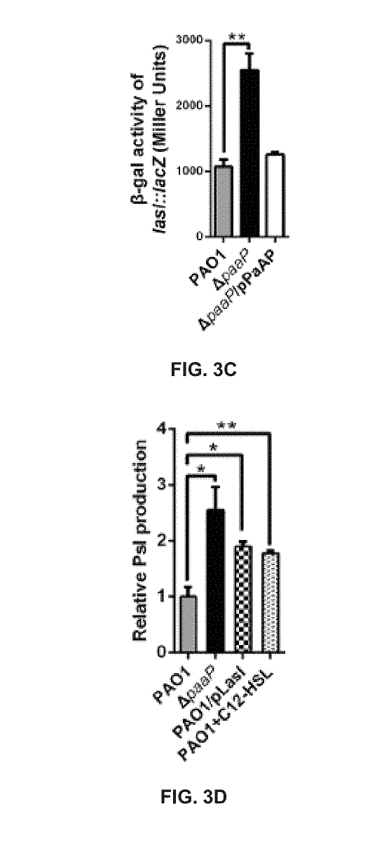

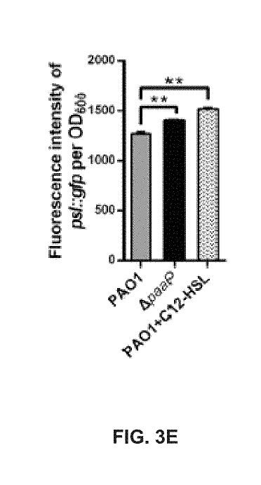

The attachment of PAO1, ΔpaaP, paap::Tn5, ΔPₚₛₗ, ΔPₚₛₗpaaP, P_{BAD}-psl, PAO1/vector, ΔpaaP/vector, ΔpaaP/pPaAP, ΔpaaP/pD308A and ΔpaaP/pPaAPNS to a surface was examined using a microtiter dish assay. The bacterial strains were inoculated on LBNS solid medium plates and incubated at 37° C. overnight. Then monoclones were inoculated in LBNS liquid medium at 37° C. overnight with shaking at 200 rpm. 50 μl of bacterial solution was inoculated in 5 ml of Jensen's medium and incubated at 37° C. with shaking at 200 rpm and 100 μl of the mid-log phase culture (OD₆₀₀˜0.5) was used for inoculation in a microtiter dish (BD Falcon). The inoculated microtiter dish was incubated at 30° C. for 30 min. Then the microtiter dish was washed. The surface-attached cells were stained with 0.1% crystal violet and solubilized in 30% acetic acid, and the OD₅₆₀ was measured. As illustrated in FIG. 3A, the corresponding anti-Psi immunodot blotting is shown under each strain. The aminopeptidase activity (AP activity) in the culture supernatant of each corresponding strain was also determined. The results were normalized to that of PAO1 (=3.6 nmol nitroanilide/OD₆₀₀). The corresponding anti-Psi immunodot blotting and AP activity were shown under each strain. The amount of Psl was normalized to the level of PAO1 (=35 μg/ml). To induce transcription of paaP, 2% arabinose was added to Jensen's medium. For the biofilm assays with an incubation time of more than 1 hour, a 1/100 dilution of a saturated P. aeruginosa culture in Jensen's medium was used for inoculation. The aminopeptidase (AP) activity was tested according to the aminopeptidase assay described previously. As shown in FIG. 3A, the ΔpaaP mutant lacked aminopeptidase activity in its culture supernatant. The ΔpaaP mutant showed significantly enhanced attachment compared with PAO1 as shown in FIG. 3A, which was consistent with the result of the paaP Tn5 insertion mutant as shown in FIG. 3A. The enhanced attachment of ΔpaaP was restored to the wild type level by expressing an intact paaP gene from the plasmid (pPaAP) as illustrated in FIG. 3B. These results indicate that the expression of PaAP influences the initial attachment of P. aeruginosa.

The ΔPₚₛₗΔpaaP mutant showed little attachment on wells of the microtiter dish as that of the Psl-negative strain WFPA800 (ΔPₚₛₗ) as illustrated in FIG. 3A. This result suggests that the enhanced surface adherence of ΔpaaP is mainly dependent on Psl, yet Psl does not affect the aminopeptidase catalytic activity of extracellular PaAP (as shown in FIG. 2A). This example illustrates that the loss of PaAP enhances the initial attachment of P. aeruginosa by increasing Psl production, and that the expression of PaAP inhibits Psl synthesis.

### Example 4

### The Effect of PaAP on the Psl Production of PAO1

To further investigate whether the aminopeptidase catalytic activity of PaAP was required to modulate Psl production, one of the key catalytic amino acids of PaAP was mutated to generate PaAP^{D308A}. The initial attachment, the Psl production, the aminopeptidase activity and the extracellular PaAP of PAO1/vector, ΔpaaP/vector, ΔpaaP/pPaAP, ΔpaaP/pD308A, ΔpaaP/pPaAPNS were measured by methods described in Example 3. The amount of Psl was normalized to the level of PAO1 (=35 μg/ml) or PAO1/vector (=28 μg/ml). AP activity was normalized to that of PAO1/vector (3.2 nmol nitroanilide/OD₆₀₀). The PaAP expression in corresponding planktonic culture detected by using an anti-PaAP antibody (normalized to that of PAO1/vector=1.9 μg/mg total extracellular protein) was shown under each corresponding column. The PaAP^{D308A} expressed from the plasmid pD308A completely lost aminopeptidase catalytic activity and could not complement the phenotype of ΔpaaP, including Psl production and surface attachment (as shown in FIG. 3B). The PaAP that lacked the signal peptide (PaAPNS) was neither able to complement the phenotype of ΔpaaP (FIG. 3B). However, all phenotypes of the ΔpaaP strain could be recovered by wild type PaAP. Given that PaAP and its mutants have similar expression levels indicated by the anti-PaAP western blotting result, these results indicated that PaAP functions extracellularly and that its aminopeptidase catalytic activity is critical to inhibit the production of Psl.

### Example 5

### The Inhibiting Effect of PaAP on the Psl Production Via Transcriptional Regulation

The transcription of lasI was tested, which was reported to affect the biofilm and the carbonhydrates production in biofilm matrix. The transcription of lasI in ΔpaaP was 3-fold higher than that in PAO1 and this phenotype could be complemented by PaAP expressed from plasmid (as shown in FIG. 3C). Expressing lasI in PAO1 also increased Psl production as that in ΔpaaP (as shown in FIG. 3D), suggesting that lasI is the factor in ΔpaaP to affect Psl production.

LasI directs the synthesis of the QS signal molecular, N-(3-oxododecanoyl)-L-homoserine lactone (C12-HSL). Thus, whether C12-HSL supplement could affect Psl production in PAO1 was examined. Indeed, addition of C12-HSL in culture medium did increase Psl production in PAO1 (FIG. 3D). C12-HSL is usually associated with the transcriptional regulator LasR to active their target genes.

Furthermore, psl transcription was enhanced by addition of C12-HSL molecular into PAO1 culture (as shown in FIG. 3E). This example shows that the QS signal molecular LasI-activated extracellular enzyme, PaAP in turn represses the transcription of lasI and leads to the inhibition of psl transcription.

### Example 6

### The Effect of Lacking PaAP on Biofilm Disruption

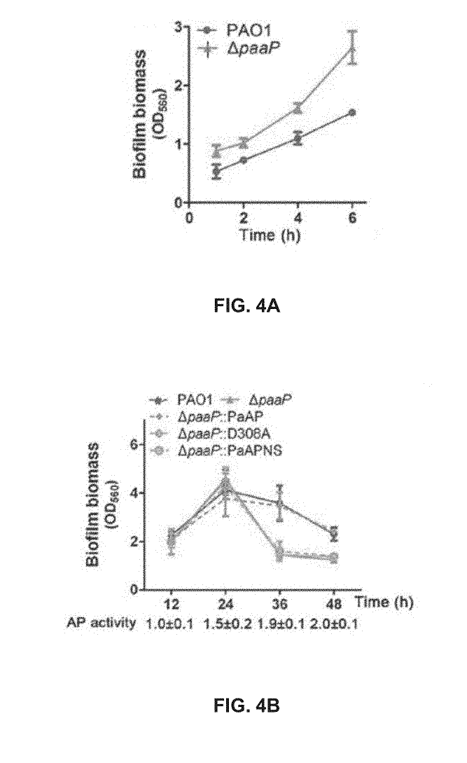

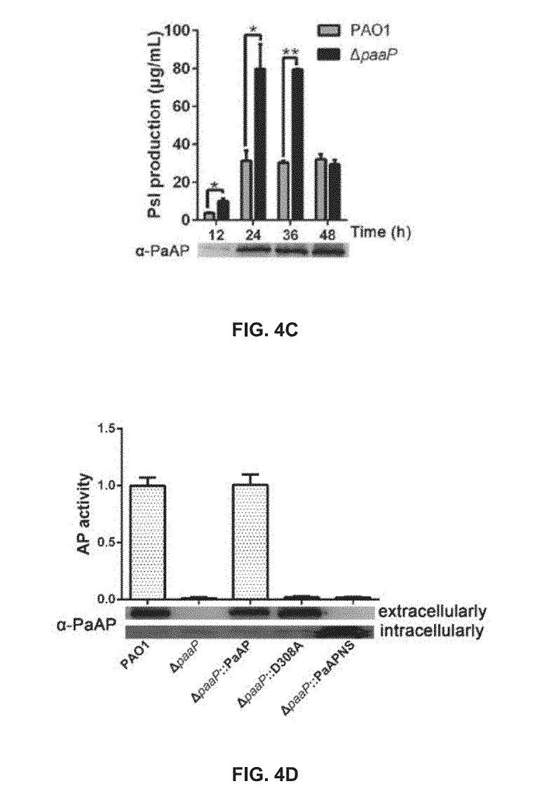

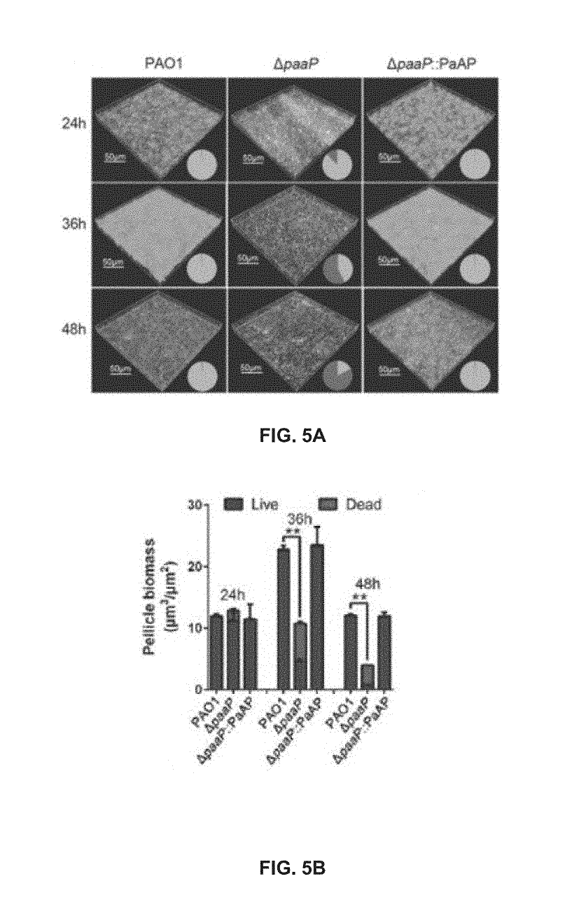

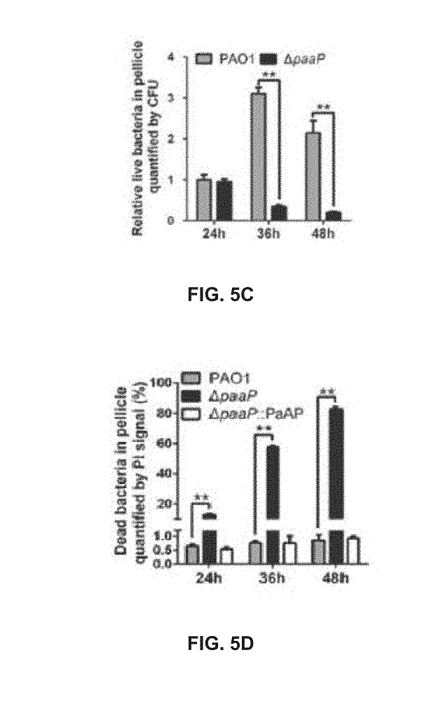

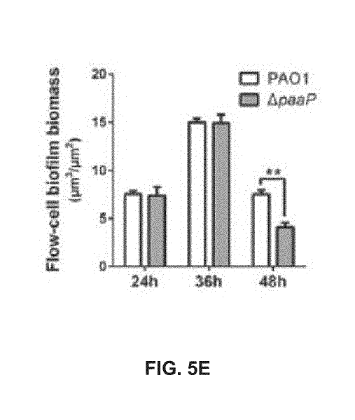

Biofilms of PAO1, ΔpaaP, paap::Tn5, ΔPₚₛₗ, ΔPₚₛₗpaaP, P_{BAD}-psl, PAO1/vector, ΔpaaP/vector, ΔpaaP/pPaAP, ΔpaaP/pD308A and ΔpaaP/pPaAPNS were grown and detected as described elsewhere in the present invention. Consistent with Psl production, the biofilm biomass of ΔpaaP mutant was higher than that of PAO1 during the first 6 hours of growth post-inoculation in a microtiter dish biofilm assay (as shown in FIG. 4A). However, the biofilm biomass of ΔpaaP decreased to the PAO1 level after 12 h and 24 h of growth (as shown in FIG. 4B). More strikingly, the biofilm biomass of ΔpaaP mutant decreased dramatically after 36 h and 48 h of growth, but PAO1 biofilms exhibited only a slight decline at these time points. Similar phenomenon of biofilm disruption was also found in the air-liquid interface biofilms (also termed as pellicle) of ΔpaaP mutant (as shown in FIG. 5B), while the ΔpaaP biofilm grown in a flow-cell with continuous media flow did not show significant disruption until 48-h of growth (as shown in FIG. 5E).

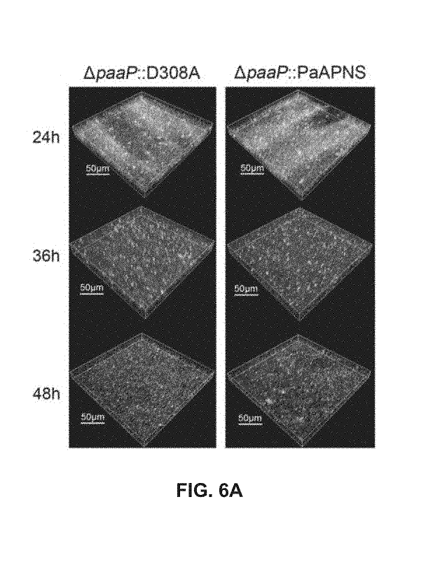

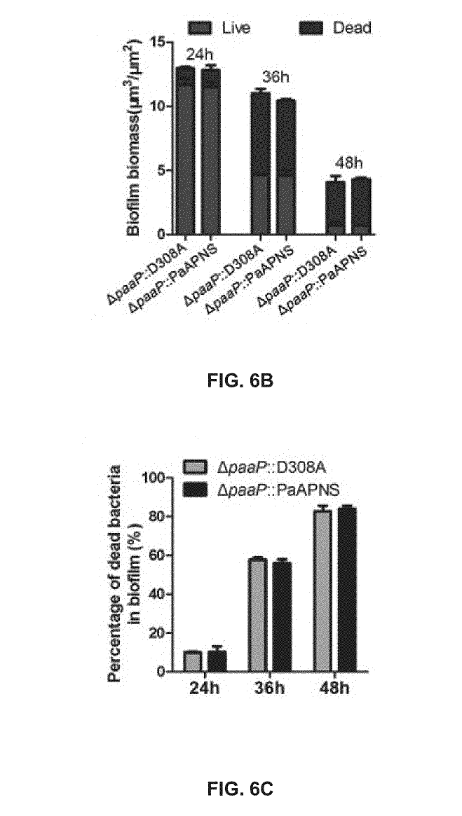

The disruption of ΔpaaP biofilms was not a result of bacterial growth because ΔpaaP displayed a similar growth rate in planktonic culture as that of PAO1 during 60 h of growth (as shown in FIG. 2). The ΔpaaP biofilm disruption was neither likely due to any deficiency in Psl synthesis because Psl extracted from the planktonic culture of ΔpaaP mutant was not less than that of PAO1 even after 48 h of growth (FIG. 4C). Biofilm disruption was also found in strains that paaP had either the PaAP active site mutation D308A or a signal peptide truncation (ΔpaaP::D308A and ΔpaaP::PaAPNS in FIG. 4B and FIG. 6B). The biofilm phenotype of the PaAP-negative strains could be complemented by wild type PaAP expressed from a chromosome-inserted copy of paaP (FIG. 4B and FIG. 5B). These results suggest that an active extracellular PaAP is important for maintaining the biofilm biomass during late stages of biofilm development. Shown in FIG. 4B under each time point was the corresponding catalytic activity of PaAP within PAO1 biofilms (the value was normalized to the level of 12 h sample, which was 2.4 nmol nitroanilide/OD₆₀₀). In FIG. 4C, the PaAP detected in corresponding supernatant of PAO1 planktonic culture was shown in the bottom (α-PaAP). Consistently, the catalytic activity of PaAP in the PAO1 biofilms increased from 12 h to 48 h and the highest activity was found at 48 h (FIG. 4B), which was consistent with the amount of PaAP detected in culture supernatant of PAO1 (FIG. 4C). When the PaAP-negative strains had the lowest biofilm biomass, the wild type had the highest aminopeptidase catalytic activity (FIG. 4B). The extracellular PaAP activity and the extracellular/intracellular presence of the α-PaAP of PAO1, ΔpaaP, ΔpaaP::PaAP, ΔpaaP::D308A and ΔpaaP::PaAPNS were measured and illustrated in FIG. 4D. The PaAP activity of ΔpaaP, ΔpaaP::D308A and ΔpaaP:PaAPNS was not detected extracellularly, while the PaAP activity of ΔpaaP::PaAP was similar to the wild type PAO1. The α-PaAP of PAO1, ΔpaaP:PaAP, ΔpaaP::D308A was detected extracellularly, while the α-PaAP of ΔpaaP:PaAPNS was detected intracellularly. The α-PaAP of ΔpaaP was not detected extracellularly nor intracellularly. This example reveals that the absence of PaAP causes the biofilm disruption in PaAP-negative strains and the catalytic activity is essential for the effect of PaAP on biofilm development.

### Example 7

### The Effect of Lacking PaAP on Bacterial Cell Death During Late Stages of Biofilm Development

The pellicles of PAO1 and ΔpaaP grown on standing culture were examined. The live/dead staining was performed to quantify the proportions of live and dead bacteria in the pellicles. Most strikingly, the ΔpaaP biofilm had a large number of dead bacteria (the PI-stained bacterial cells with a compromised cell membrane were considered to be dead in this live/dead method) compared with PAO1 biofilm (FIG. 5A and FIG. 5B). The proportion of dead bacteria in the ΔpaaP biofilm was 13% at 24 h, increasing to 58% at 36 h, and 83% at 48 h (FIG. 5D). In contrast, the dead bacteria in wild type biofilm were less than 1% at the three time points. To rule out the possibility that the absence of PaAP might affect bacterial permeability and result in inaccurate quantification of the dead bacteria, the pellicles were dispersed and then the live bacteria were quantified on the basis of the number of CFUs. The CFU calculation showed similar portion of live bacteria as the quantitation of live/dead staining (FIGS. 5B and 5C). The values of live bacteria in pellicles shown in FIG. 5C were normalized to the level of 24 h PAO1 sample, which was 9.3×10⁷ CFU/ml.

The pellicles of PaAP-negative strains containing either the D308A mutation or signal peptide truncation also showed similar amounts of dead bacteria as ΔpaaP (FIG. 6). Furthermore, the bacterial cell death in the pellicles of the three PaAP-negative strains could be complemented by expression of paaP from its own promoter (FIG. 5A to 5C and FIG. 6). These data suggested that the absence of PaAP within biofilms led to bacterial cell death during late stages of biofilm development, which might be the reason that caused biofilm disruption.

### Example 8

### The Effect of PslG Released from Dead Bacteria on the Disruption of the Psl Matrix

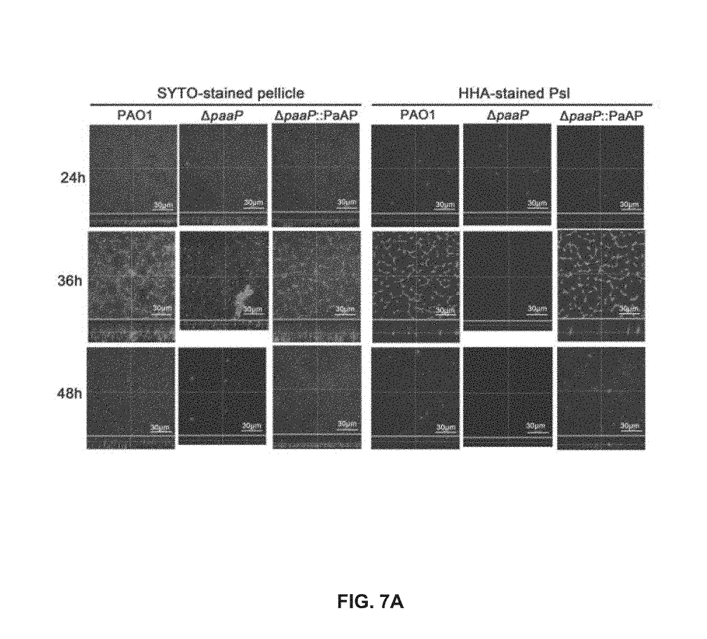

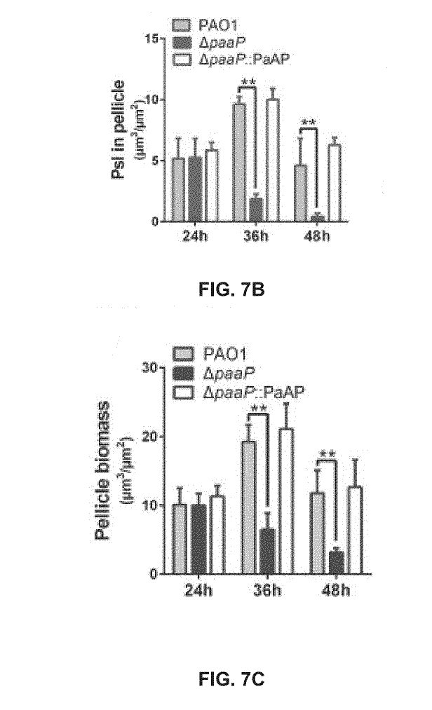

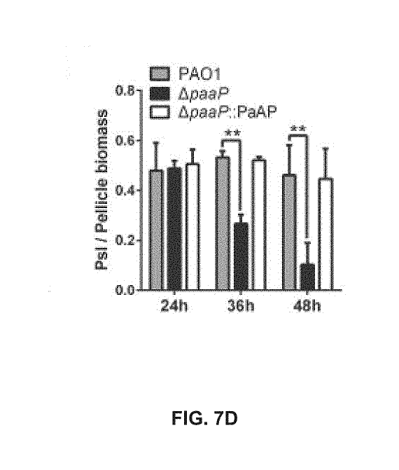

Psl in pellicles was detected by lectin HHA (Hyppeastrum hybrid lectin from amaryllis) staining. Meanwhile, SYTO9 was used to stain pellicle bacteria (both live and dead) (FIG. 7A). The pellicles of ΔpaaP had a similar Psl matrix compared with PAO1 after 24 h of growth. However, the Psl in biomass of 36-h ΔpaaP pellicles was reduced by 60%, whereas the Psl of 36-h PAO1 doubled its biomass and showed a clear fiber matrix (FIG. 7A, B). For the 48-h pellicles, little Psl was detected in ΔpaaP pellicles (less than 10% of the 24-h pellicle) (FIG. 7B), while Psl of PAO1 pellicles returned to a level similar to that of 24 h. The amount of Psl in pellicles was consistent with the pellicle biomass at the corresponding time point, thus supporting the importance of Psl for maintenance of the biofilm biomass (FIG. 7B, C). Given that the ΔpaaP mutant had the ability to synthesize Psl and produced even more Psl than PAO1 (FIG. 4C), these results suggested that Psl degradation might have occurred in biofilms of the ΔpaaP mutant.

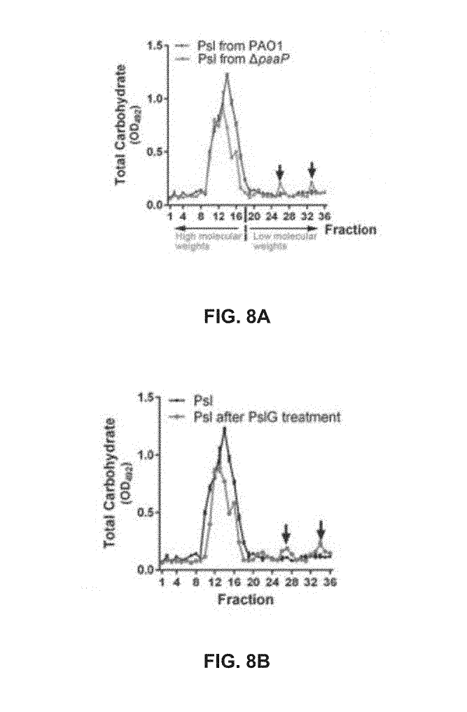

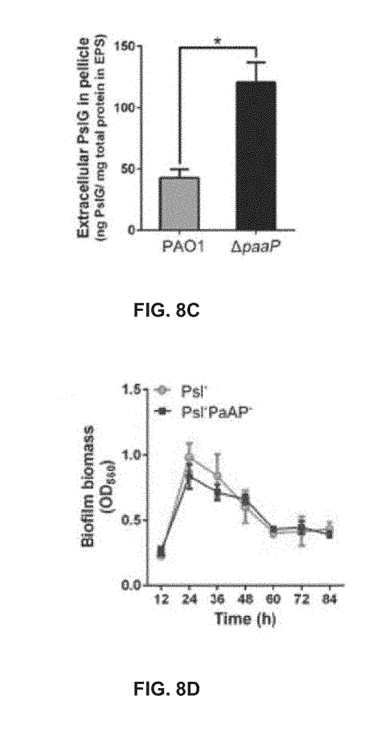

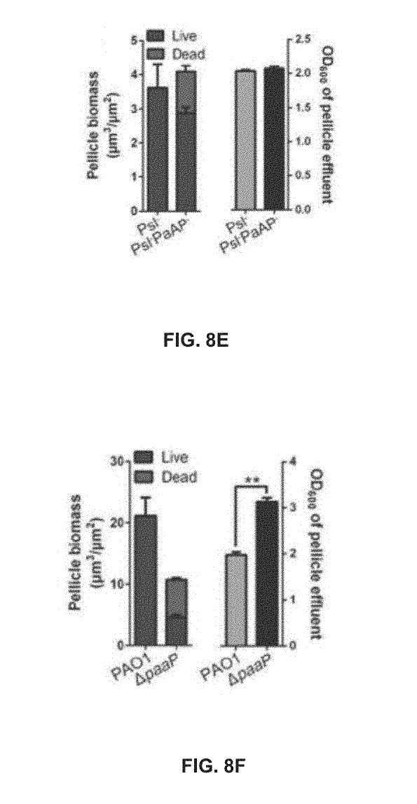

To examine Psl degradation within biofilms, Psl exopolysaccharide was extracted from 48-h PAO1 and ΔpaaP biofilms respectively. The Psl extracts were run on a Sephadex G-50 column to fractionate exopolysaccharide polymers according to their molecular weights. The elution profiles showed that the high-molecular-weight Psl was decreased in ΔpaaP compared with PAO1 biofilms (as shown in FIG. 8A). In addition, two new peaks appeared in the low-molecular-weight region in the ΔpaaP samples (as shown in FIG. 8A, indicated by arrows). This result showed that there was Psl degradation occurring in the ΔpaaP biofilms.

The extracellular PslG in biofilm matrices was examined by using an anti-PslG antibody. The extracellular PslG extracted from the 48-h ΔpaaP pellicles was 3-fold higher than that from PAO1 pellicles (FIG. 8C). This result suggests that extracellular PslG released from dead bacteria is probably the cause of Psl matrix disruption.

To confirm, the Psl extracted from PAO1 biofilms was treated with exogenous-supplied PslG in vitro. Strikingly, PslG-degraded Psl showed a very similar profile as Psl extracted from the ΔpaaP biofilm in a Sephadex G-50 column (FIG. 8B). These results indicate that Psl degradation occurs in the ΔpaaP biofilm and suggests that disruption of ΔpaaP biofilms is due to the degradation of Psl by extracellular PslG, thereby leading to biofilm dispersion.

To further investigate the contributions of Psl on the bacterial cell death in ΔpaaP biofilms and the link among bacterial cell death, Psl degradation and biofilm disruption, the Psl and PaAP double mutant strain, Psl-PaAP-(ΔPₚₛₗΔpaaP) that could not synthesize the exopolysaccharide Psl, PaAP, and PslG was utilized. Biofilm formation by the Psl⁻PaAP⁻ was similar to that of Psl-negative strain within 84 h of growth in a microtiter dish (FIG. 8D). However, there were 100-fold more dead bacteria in pellicles of the Psl⁻PaAP⁻ than that of Psl-negative strain (containing only 0.4% dead bacteria) (FIG. 8E, left panel). Given that no biomass reduction was found for the Psl⁻PaAP⁻ biofilms (FIG. 8D, E) despite the presence of large amounts of dead bacteria (FIG. 8E, left panel), this result suggests that the reduction of biofilm biomass in the ΔpaaP strain is mainly due to Psl degradation-related dispersion and that Psl had little contribution on bacterial cell death in ΔpaaP biofilms. The proportion of dead bacteria in either ΔpaaP or Psl⁻PaAP⁻ biofilm was comparable (FIGS. 8E and F, left panel). However, the live bacteria in ΔpaaP pellicles were reduced proximately by 80% compared to PAO1 pellicles whereas the live bacteria in Psl⁻PaAP⁻ pellicles had little reduction compared with Psl (FIGS. 8E and F, left panel).

The OD₆₀₀ of the pellicles' effluent from PAO1, ΔpaaP, Psl⁻, and Psl⁻PaAP⁻ was examined. OD₆₀₀ of the effluent from ΔpaaP pellicles showed a 50% increase compared to PAO1 (FIG. 8F, right panel), which was in agreement with the biomass reduction of the ΔpaaP pellicle (reduced by approximately 50% of the PAO1 level) (FIG. 8F, left panel). On the contrary, no significant differences were found for the OD₆₀₀ of pellicle effluents between Psl⁻ and Psl⁻PaAP⁻ strains (FIG. 8E, right panel). Taken together, this example suggested that the disruption of ΔpaaP biofilms was a result of the degradation of Psl matrix by PslG released from the dead bacteria within biofilms, which in turn led to the dispersion of live bacteria from biofilms into effluents.

### Example 9

### The Effect of Lacking PaAP on the Sensitivity of P. aeruginosa to Ciprofloxacin

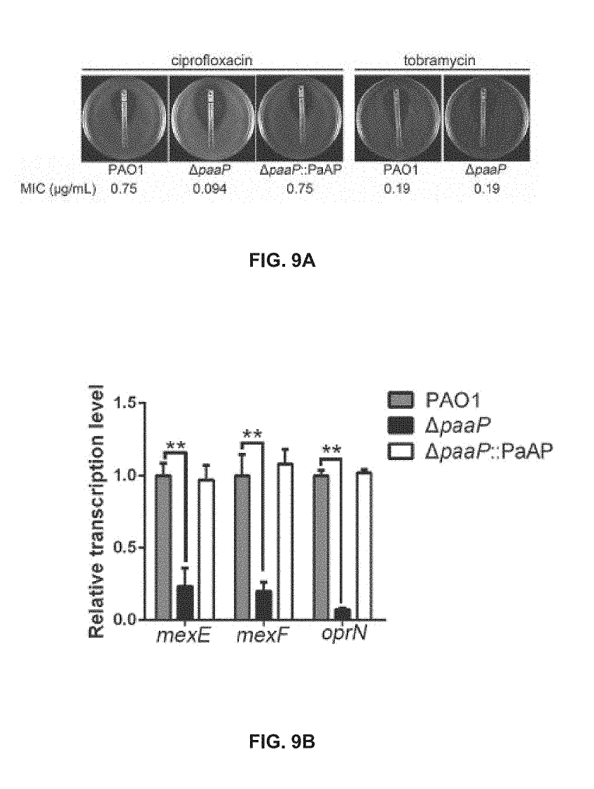

The MIC (minimum inhibition concentration) of ΔpaaP strain to tobramycin and ciprofloxacin, two antibiotics commonly used to treat P. aeruginosa infections, was tested by E test strips. Overnight culture was diluted to OD₆₀₀˜0.1, and spread on the plate. The E test strip was put onto the plate, cultivated at 37° C. overnight, and the minimum inhibition concentration was recorded according to the E test strip. The ΔpaaP showed similar MIC of tobramycin as that of PAO1, yet its MIC to ciprofloxacin was 8-fold lower than PAO1 (as shown in FIG. 9A). The aminopeptidase active site mutation D308A (ΔpaaP::D308A) or a signal peptide truncation (ΔpaaP::PaAPNS) exhibited a similar MIC as that of ΔpaaP.

The MexEF-oprN multidrug pump plays a role in the resistance of P. aeruginosa to ciprofloxacin, and thus the expression of mexEF-oprN multidrug efflux operon was examined by quantitative real-time PCR. Transcription of mexEF and oprN in ΔpaaP were 4-6 fold lower than that of PAO1 and ΔpaaP::PaAP strain (FIG. 9B). These results indicates that lacking PaAP or its aminopeptidase activity enhance the sensitivity of P. aeruginosa to ciprofloxacin.

### Example 10

### The Effect of the Loss of an Aminopeptidase on the Biofilm of P. stutzeri

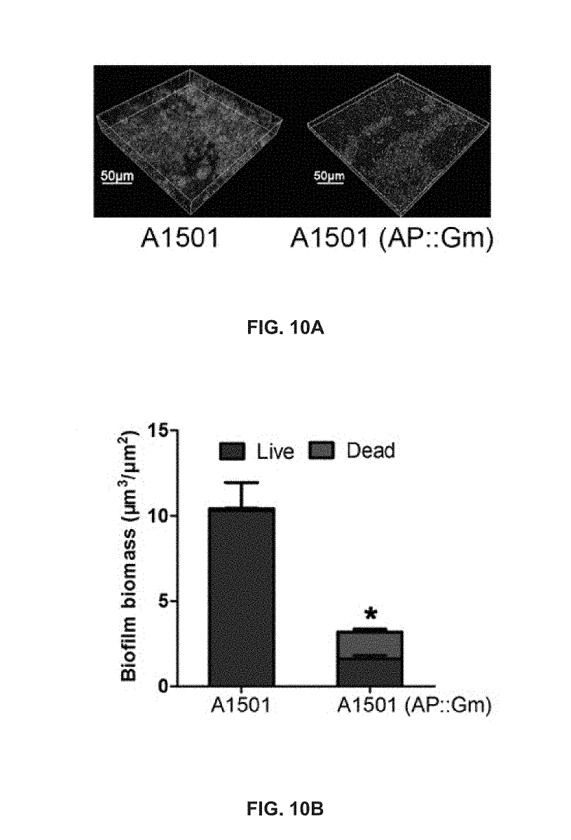

The pellicles of P. stutzeri (A1501) and an aminopeptidase mutant A1501 (AP::Gm) grown on standing culture were examined to evaluate the effect of the loss of an aminopeptidase on the pellicles of P. stutzeri. The live/dead staining was performed as described previously in the present invention to quantify the proportions of live and dead bacteria in the pellicles. The results were shown in FIG. 10. The biofilm biomass of A1501 (AP::Gm) was significantly lower than that of the wild type A1501. Approximately half of the bacterial cells of A1501 (AP::Gm) were dead. These results were similar to the results of PAO1 and ΔpaaP (as shown in FIG. 5). This example indicates that the aminopeptidase also plays a similar role in the biofilm formation of P. stutzeri as in P. aeruginosa.

### Example 11

### The Effect of PaAP on Biofilm Formation and Environmental Adaption of P. aeruginosa

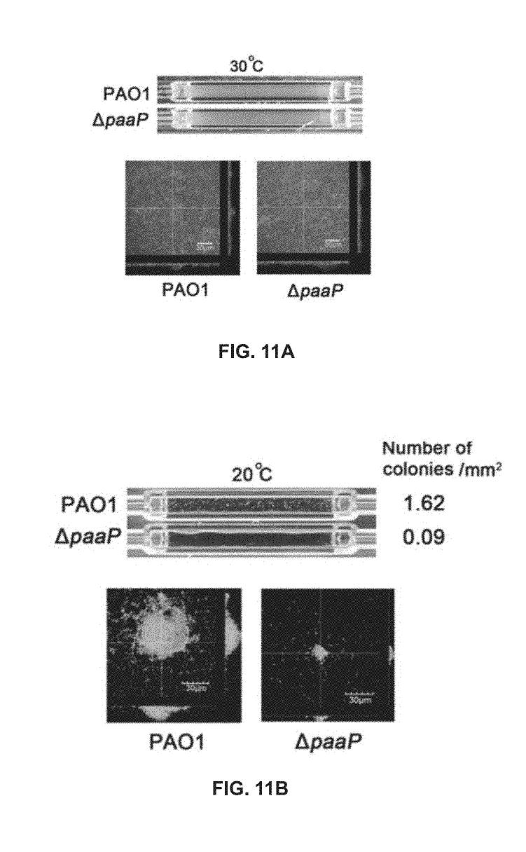

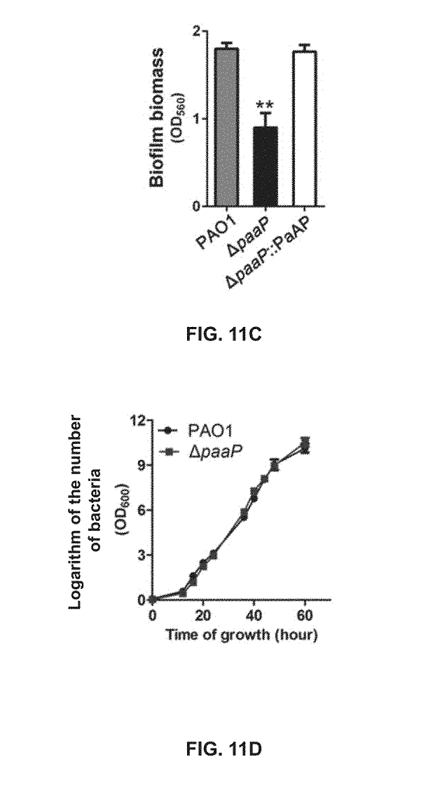

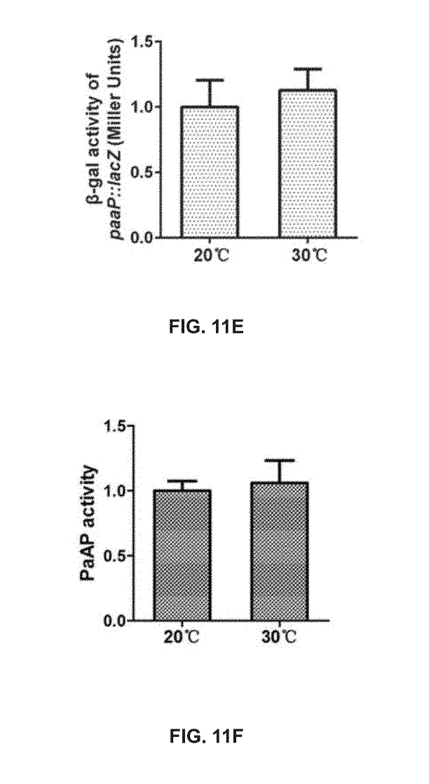

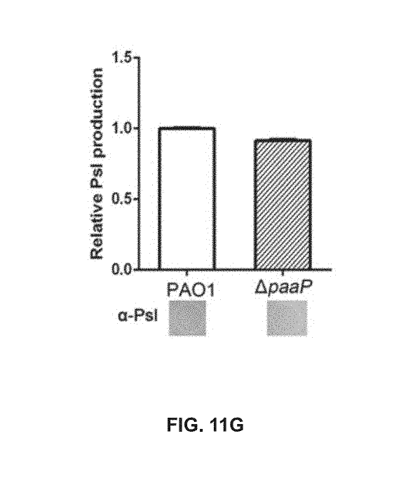

The biofilms of PAO1 and ΔpaaP were grown at 30° C. and 20° C. for 24 h in a flow cell system. Photos of the biofilms of PAO1 and ΔpaaP were taken by using a camera. The biofilms were stained by SYTO9 and the images were acquired by the CLSM. The number of colonies per square millimeter was calculated for the biofilms of PAO1 and ΔpaaP grown at 20° C. The biofilm biomass of PAO1 and ΔpaaP grown at 20° C. after 24 h in the flow cell system (FIGS. 11A, 11B) and in microtiter dish (FIG. 11C) was measured as previously described in the present disclosure. These results are shown in FIGS. 11A, 11B and 11C. As shown in FIGS. 11A and 11B, the PAO1 formed flat biofilms with some microcolonies in a flow-cell chamber after 24 h of growth at 30° C., while the PAO1 mainly formed microcolonies at 20° C. under the same growth conditions and ΔpaaP could only form a few microcolonies at 20° C. (the number of microcolonies for was 180-fold less than that of PAO1). Consistently, the 24-h biofilm biomass of ΔpaaP at microtiter dish assay was also significantly lower than that of PAO1 grown at 20° C. (FIG. 11C). Growth curves of PAO1 and ΔpaaP under the same growth conditions at 20° C. were generated by measuring the absorbance at 600 nm of the bacterial planktonic culture at different time points using a spectrophotometer. Results are shown in FIG. 11D. No significant difference in the growth of PAO1 and ΔpaaP was observed. The PaAP transcription level and the PaAP acrivity in PAO1 at 20° C. and 30° C. were measured. The β-Galactosidase activity of paaP::lacZ was normalized to the value of PAO1 grown at 20° C. (=120.27 Miller Units) and corresponding results are shown in FIG. 11E. The PaAP activity was normalized to the value at 20° C. (=2.9 nmol nitroanilide/OD₆₀₀) and corresponding results are shown in FIG. 11F. As illustrated by FIGS. 11E and 11F, no significant difference was observed for the expression of PaAP and the PaAP activity between 20° C. and 30° C. The Psl produced by PAO1 and ΔpaaP in planktonic culture at 20° C. was measured as previously described in the present invention. The amount of Psl was normalized to that of PAO1 (=15 μg/ml) and the results are shown in FIG. 11G. The corresponding anti-Psi immunodot blotting is shown under each column. No significant difference was observed for the production of Psl in PAO1 and ΔpaaP in planktonic culture at 20° C.

Therefore, this example suggests that PaAP benefits the biofilm formation and environmental adaptation of P. aeruginosa at low temperature, which may not rely on the production of Psl.

### Example 12

### The Inhibition Effect of Amastatin on the Enzyme Activity of PaAP

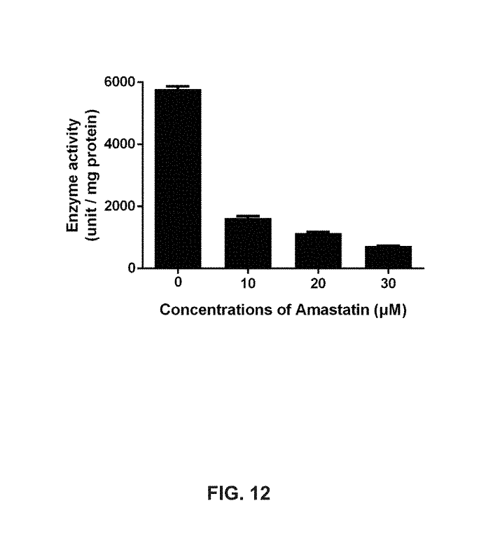

The PaAP catalytic activity was determined according to Sarnovsky's method. Purified PaAP was used for the assay. Equal volumes of substrate Leu-p-nitroanilide solution (1.2 mM Leu-p-nitroanilide in 0.1 M Tris-HCl, 2 mM CaCl₂, pH 8.3) were added to 7 nM PaAP solution with 10 μM, 20 μM, 30 μM of Amastatin or without Amastatin. Then the mixture was incubated at 37° C. for 1 hour. A molar extinction coefficient of 10400 for p-nitroaniline was used to calculate the amount of product released from nitroanilide (NA)-based substrates. PaAP catalytic activity was calculated in nmol of nitroanilide per mg PaAP. As illustrated in FIG. 12, the results suggest that the Amastatin may inhibit the enzyme activity of PaAP at micromolar concentrations, and the inhibition effect may increase with higher concentration of Amastatin.

Therefore, this example illustrates that the aminopeptidase inhibitor Amastatin may inhibit the enzyme activity of PaAP and may be a potential anti-biofilm agent targeting PaAP.

### Example 13

### The Use of PslG to Disperse Bacterial Aggregates or Biofilms for Bacterial Cell Counting by OD Measurement or Living Cell Counting by CFU

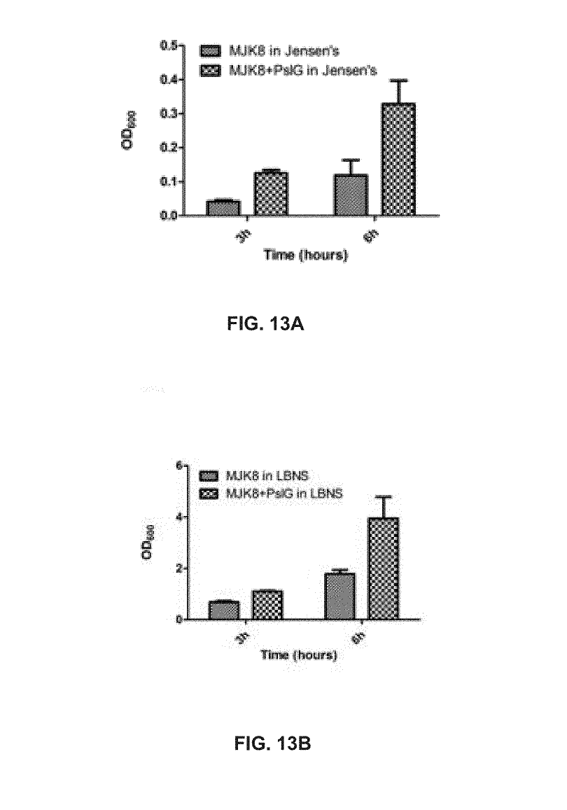

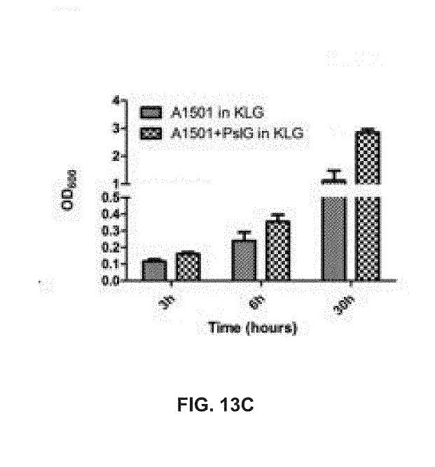

PAO1-derived rugose small colony variant (RSCV) strain MJK8 was grown in LBNS at 37° C. 200 rpm overnight. This overnight culture was washed and then inoculated into Jensen's medium or in LBNS medium at 1:50 with or without 100 nM of PslG. Bacterial growth was determined by taking OD₆₀₀ read at 3 h and 6 h post inoculation. P. stutzeri strain A1501 was firstly grown in LD medium at 30° C. 200 rpm overnight. The overnight culture was washed and inoculated into KLG⁺ medium at 1:50 with or without 100 nM of PslG. OD₆₀₀ was taken at 3 h, 6 h and 30 h post inoculation to monitor the bacterial growth. FIGS. 13A-13C show the comparison of OD₆₀₀ read with or without PslG treatment of: MJK8 growth in chemically defined Jensen's medium (FIG. 13A); MJK8 growth in LBNS rich medium (FIG. 13B); A1501 growth in KLG⁺ medium (FIG. 13C). As illustrated in FIGS. 13A-13C, PslG can disperse the flocs of MJK8 and A1501 in liquid culture and facilitate the measurement of bacterial numbers at OD₆₀₀.

To enumerate live bacteria in the biofilms formed by P. aeruginosa PAO1 strain and PAO-derived PaAP mutant strain ΔpaaP, biofilms formed in Jensen's medium were washed with PBS and dispersed with 50 nM PslG at 30° C. for 30 minutes, and the number of CFUs was detected accordingly. Results are shown in FIG. 5C. The results suggest that the ratio of live bacteria count after the PslG treatment is comparable to the quantification from the live/dead staining results (FIG. 5B). This example shows that PslG may be used to disperse bacterial aggregates or biofilms for bacterial cell counting by OD measurement or live cell counting by CFU.

### Example 14

### Results of Using PslG or Tissue Homogenate to Disperse Biofilms and Results of Live Cell Counting by CFU

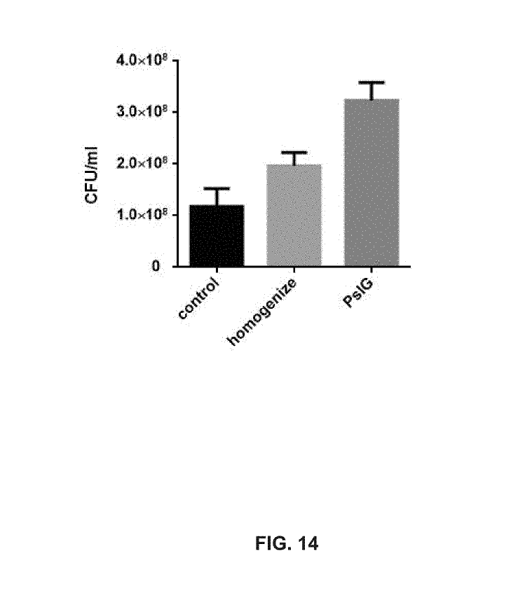

The PAO1 strain was inoculated on LBNS solid medium plates and overnight incubated at 37° C. Then monoclones of the PAO1, collected from the LBNS solid medium plates, were inoculated in LBNS liquid medium and incubated at 37° C. overnight with shaking at 200 rpm. The culture was washed with PBS for once. Then the culture was inoculated in Jensen's liquid medium at 1:100 (V:V), and incubated at 30° C. in a 24-pore plate. Biofilms were formed at the air-liquid interface. The liquid under the biofilms was removed by a pipettor. The biofilms were washed with PBS for twice so as to remove free bacterial cells that are not embedded in the biofilms. The biofilms were added in PBS for subsequent treatment. For a tissue homogenate treatment group, a sterile glass homogenizer (1 mL) was used to homogenize the biofilms for three times. For PslG treatment group, PslG was added to the biofilms, and the final concentration of PslG is 50 nM. The biofilms were incubated at 30° C. for 1 h. For a control group, no treatment was performed on the biofilms at this stage. Afterwards, the biofilm samples from the tissue homogenate treatment group, the PslG treatment group, and the control group were homogenized using a pipette and then votexed. The biofilm samples were diluted in PBS at 1:10. The diluted biofilm samples were then inoculated on LB solid medium plates and incubated at 37° C. overnight. The numbers of live cells in the biofilm samples was determined based on the CFUs. As shown in FIG. 14, the number of detected CFUs for biofilms treated by PslG was significantly greater than the number of detected CFUs for the biofilms treated by tissue homogenate, and the biofilms in the control group.

This example shows that the PslG treatment has a better effect in dispersing the biofilms to detect the number of live cells in the biofilms, compared with the tissue homogenate treatment.

### Example 15

### Results of Using PslG or Tissue Homogenate to Disperse Bacteria Aggregates in the Suspension of Bacteria that Tend to Aggregate (or Flocculate)

The MJK8 strain was inoculated on LBNS solid medium plates and overnight incubated at 37° C. Then monoclones of the MJK8, collected from the LBNS solid medium plates, were inoculated in LBNS liquid medium and incubated at 37° C. overnight with shaking at 200 rpm. The culture was washed with PBS for once. The culture was then incubated in Jensen's liquid medium at 1:50 (V:V). For a PslG treatment group, the Jensen's liquid medium contains 25 nM of PslG. The culture was incubated at 37° C. with shaking at 200 rpm. Samples of the culture were obtained every 3 h in 24 h for detecting the OD₆₀₀ value and the concentration of total proteins. For a homogenate group, the culture was incubated at 37° C. with shaking at 200 rpm. Samples of the culture were obtained every 3 h in 24 h. The samples were homogenized using a glass homogenizer for three times. Then the OD₆₀₀ value and the concentration of total proteins were detected for the homogenized samples. For a control group, the culture was incubated at 37° C. with shaking at 200 rpm. Samples of the bacteria solution were obtained every 3 h in 24 h for detecting the OD₆₀₀ value and the concentration of total proteins.

In the above steps, the concentration of total proteins was determined using a BCA (bicinchonininc acid) method. 100 μl of bacteria solution from the samples was centrifuged to collect the bacteria. The collected bacteria were washed with 100 μl PBS for once. The bacteria were re-suspended in 100 μl of PBS. A container including the bacteria suspension was repeatedly put in liquid nitrogen and water bath at 37° C. for three cycles of freezing and thawing. The bacteria suspension was then centrifuged at 14000 rpm and at 4° C. for 15 minutes. The supernatant was transferred to a new centrifuge tube. 25 μl of the supernatant was used to detect the concentration of the total proteins by Pierce® BCA Protein Assay Kit. A standard curve was obtained using 0.5 mg/ml BSA (Bull Serum Albumin) proteins. An equation related to the standard curve may be obtained (Y=1.148X+0.0407, R²=0.9901). The concentration of the total proteins in all the samples may be calculated based on the equation.

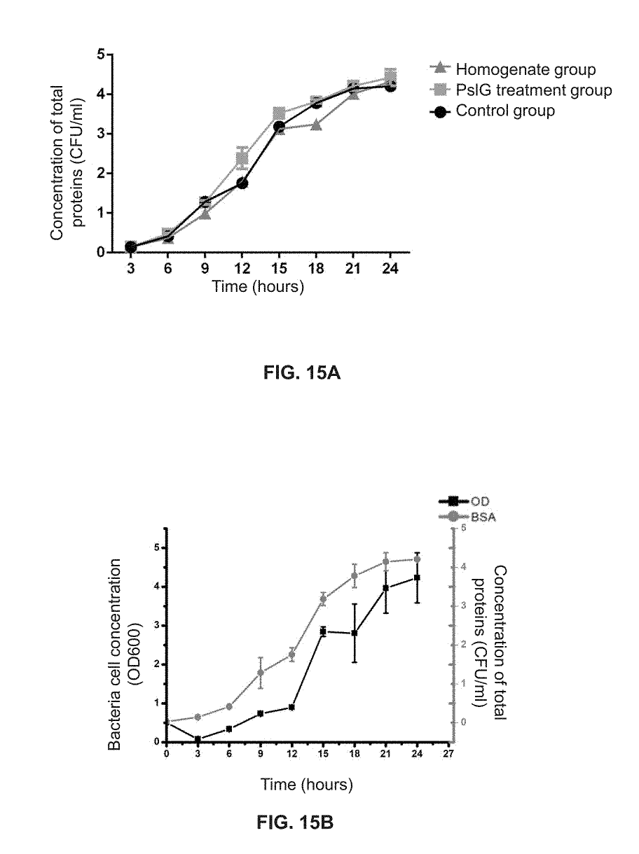

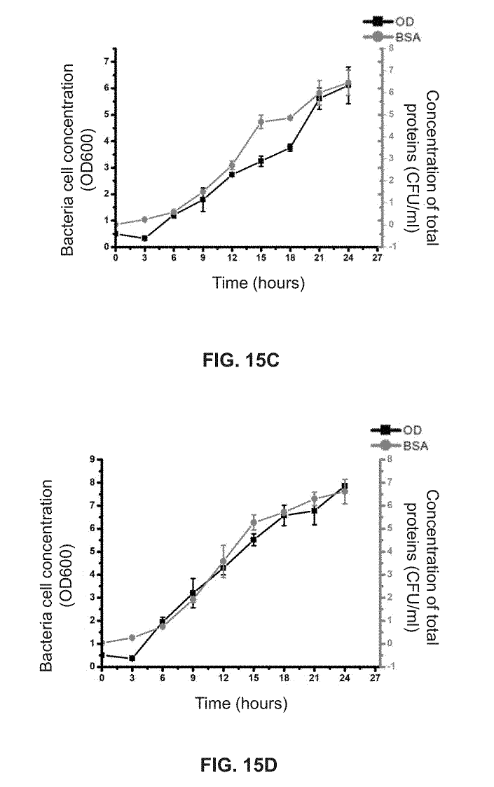

Since the OD₆₀₀ measurement may not be directly used to accurately determine the number of bacterial population for the bacteria that tend to aggregate or flocculate, the growth curve of the bacteria was obtained by detecting the concentration of total proteins in the bacteria solution. As shown in FIG. 15A, the growth curves of the control group, the tissue homogenizing group and the PslG treatment group were similar. FIGS. 15B-15D show a comparison between the growth curve obtained based on OD₆₀₀ measurement and the growth curve obtained based on the BCA method for the three groups. The two growth curves shown in FIG. 15B for the control group are significantly different. The similarity degree between the two growth curves shown in FIG. 15D for the PslG group is greater than the similarity degree between the two growth curves shown in FIG. 15C. This example shows that PslG can be added in the liquid medium of the bacteria that tend to aggregate or flocculate, and then the growth curve of the bacteria can be obtained using the conventional method based on OD₆₀₀ measurement.

It should be noted that the above examples are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

We claim:

1. A method of removing, reducing, dispersing, disrupting or eradicating biofilm present on a surface, comprising reducing potency of an aminopeptidase in the biofilm.

2. The method of claim 1, wherein to reduce the potency of the aminopeptidase, the method comprises contacting the biofilm with a composition that comprises an agent that:
reduces expression of the aminopeptidase protein in the biofilm;
reduces secretion of the aminopeptidase by bacteria in the biofilm, or
reduces extracellular activity of the aminopeptidase in the biofilm.

3. The method of claim 2, wherein the agent comprises an acid agent, an alkaline agent, or a chelating agent.

4. The method of claim 3, wherein the chelating agent comprises a zinc chelating agent that reduces zinc concentration that maintains the extracellular activity of the aminopeptidase in the biofilm, and the acid or the alkaline disrupts a pH environment that maintains the extracellular activity of the aminopeptidase in the biofilm.

5. The method of claim 2, wherein the agent comprises an inhibitor that antagonizes the aminopeptidase.

6. The method of claim 5, wherein the inhibitor is an antibody.

7. The method of claim 5, wherein the inhibitor is Amastatin or its derivative compounds.

8. The method of claim 2, wherein the agent reduces expression of the aminopeptidase protein by bacteria in the biofilm and comprises a small interfering RNA (siRNA), an antisense DNA (asDNA), an antisense RNA (asRNA), or an aptamer.

9. The method of claim 2, wherein the agent comprises a blocker that reduces the secretion of the aminopeptidase from bacteria in the biofilm by blocking a signal peptide that facilitates the secretion.

10. The method of claim 2, wherein the composition further comprises an antibiotic.

11. The method of claim 2, wherein the composition further comprises a pharmaceutically acceptable carrier.

12. The method of claim 1, wherein to reduce the potency of the aminopeptidase in the biofilm, the method comprises modifying a nucleic acid encoding the aminopeptidase by inserting one or more nucleotides, deleting one or more nucleotides, and/or replacing one or more nucleotides.

13. The method of claim 1, wherein to reduce the potency of the aminopeptidase in the biofilm, the method comprises reducing secretion of the aminopeptidase by mutating a nucleic acid encoding a signal peptide that facilitates the secretion of the aminopeptidase.

14. The method of claim 1, wherein the biofilm is formed by P. aeruginosa and the aminopeptidase has an amino acid sequence of at least 90% identity to SEQ ID NO: 1.

15. The method of claim 14, wherein the composition enhances sensitivity of P. aeruginosa to ciprofloxacin.

16. The method of claim 1, wherein the biofilm is formed by P. stutzeri and the aminopeptidase has an amino acid sequence of at least 90% identity to SEQ ID NO: 3.

17. A method of removing, reducing, dispersing, disrupting or eradicating biofilm present on a surface, comprising contacting the biofilm with a composition that comprises an agent that:
(a) reduces expression of the aminopeptidase protein in the biofilm;
(b) reduces secretion of the aminopeptidase by bacteria in the biofilm; or
(c) reduces extracellular activity of the aminopeptidase in the biofilm,
wherein the aminopeptidase has an amino acid sequence of at least 95% identity to SEQ ID NO: 1 and the biofilm is formed by P. aeruginosa, or
the aminopeptidase has an amino acid sequence of at least 95% identity to SEQ ID NO: 3 and the biofilm is formed by P. stutzeri.

18. The method of claim 17, wherein the agent comprises:
(a) an acid agent, an alkaline agent, or a chelating agent, which disrupt ion concentration and pH environment that maintain the extracellular activity of the aminopeptidase in the biofilm;
(b) an inhibitor that specifically antagonizes the aminopeptidase;
(c) an siRNA, an asDNA, an asRNA, or an aptamer, which reduce expression of the aminopeptidase protein by bacteria in the biofilm; or
(d) a blocker that reduces the secretion of the aminopeptidase by blocking a signal peptide that facilitates the secretion.

19. The method of claim 17, wherein reducing the potency of the aminopeptidase causes cell death of bacteria in the biofilm.

20. The method of claim 17, wherein reducing the potency of the aminopeptidase causes disruption of Psl matrix and thus causes dispersion of the biofilm.

21. The method of claim 17, wherein the composition further comprises an antibiotic.

22. The method of claim 21, wherein the antibiotic and the agent have synergistic effects in removing, reducing, disrupting or eradicating the biofilm or the bacterial aggregate.

23. The method of claim 21, wherein the antibiotic is ciprofloxacin or any antibiotics that kill Pseudomonas species.

24. A method of determining the number of bacterial population in biofilm or a culture containing bacterial aggregate, comprising:
(a) dispersing the bacterial aggregate or biofilm by using a composition comprising exogenous PslG; and
(b) determining the number of bacteria based on the dispersed bacteria.

25. The method of claim 24, wherein the bacteria aggregate or biofilm is formed by P. aeruginosa or P. stutzeri.


SEQUENCE LISTING (28 sequences)

SEQ ID NO: 1 - protein, 536 aa, Pseudomonas aeruginosa
MSNKNNLRYALGALALSVSAASLAAPSEAQQFTEFWTPGKPNPSICKSPLLVSTPLGLPRCLQASNVVKRLQKLEDIASLNDGNRAAATPGYQASVDYVKQTLQKAGYKVSVQPFPFTAYYPKGPGSLSATVPQPVTYEWEKDFTYLSQTEAGDVTAKVVPVDLSLGAGNTSTSGCEAEDFANFPAGSIALIQRGTCNFEQKAENAAAAGAAGVIIFNQGNTDDRKGLENVTVGESYEGGIPVIFATYDNGVAWSQTPDLQLHLVVDVVRKKTETYNVVAETRRGNPNNVVMVGAHLDSVFEGPGINDNGSGSAAQLEMAVLLAKALPVNKVRFAWWGAEEAGLVGSTHYVQNLAPEEKKKIKAYLNFDMIGSPNFGNFIYDGDGSDFGLQGPPGSAAIERLFEAYFRLRGQQSEGTEIDFRSDYAEFFNSGIAFGGLFTGAEGLKTEEQAQKYGGTAGKAYDECYHSKCDGIANINQDALEIHSDAMAFVTSWLSLSTKVVDDEIAAAGQKAQSRSLQMQKSASQIERWGHDFIK

SEQ ID NO: 2 - protein, 24 aa, Pseudomonas aeruginosa
MSNKNNLRYALGALALSVSAASLA

SEQ ID NO: 3 - protein, 534 aa, Pseudomonas stutzeri
MHKKNNAVRVVASLALIASSAALAAPKADTAQFNDFWTPAKPNAALCRSPLLVGTPVGLPRCMQASNVMKHLEALQDIATLNGGNRASGQPGYQASLDYVRSRLQRAGYRVEVQAFPFLAFYPVNPGTLSAVAPQTVQYVWEEDFAYADQTDPGNVTAPVVPVDLVLGAGNTSTSGCEPEDFAGFPAGAIALMQRGTCPFGQKATNAAAAGAAGAIIFNQGDTEDRKGLLVATLGEDYAGGIPVLFSTYDNGVAWSQTAGLQLSMNVDVVREQTETYNLLAETRRGDPSNVVMVGAHLDSVFEGAGINDNGSGSAALLEMALLMSKARPENKVRFAWWGAEESGLVGSTYYVNQLPDEQKQRIKAYLNVDMIGSPNYANFIYDGDGSDFGLQGPPGSAAIERLLRTYFKLRNAPSEGTEIDFRSDYAQFFEDGIAFGGLFTGAEDIKTEEQAQRYGGTAGQSFDQCYHTPCDNLGNISIEALELHGDALAFATSWLSLSTKMIDDEIAAAAEQSIGTMRIQQVQEKSRWGHWIR

SEQ ID NO: 4 - protein, 24 aa, Pseudomonas stutzeri
MHKKNNAVRVVASLALIASSAALA

SEQ ID NO: 5 - DNA, 1329 bp, Artificial sequence
  Synthetic sequence
atggcacgtaagggactctatctgggcggcagcgcgctgctgctcgccgtggtactgctgctggtgttctgggggcgtcccgccgacgccgagatccaggtactgaaggcgcctcgcgcggtggtctggaaagacttcctcggggtcaacgcgcagttcctctggttcagcccggagcgttacaacaagcagatcgaccgcctgcaggacctggggctggagtgggtgcgcctggacctgcactgggaccgcctggaaaccgccgaggaccagtaccagctggcctccctcgaccagttggtcaaagatctcgaggcgcgccagctgaagtcggtgttctacctggtcggctcggcccgcttcatcaccaccgcgccgttctactcgcccttccaggaccagtatccgccgcgcgacccggaagtcttcgcccggcgcatggcgatgctctcgcagcgctacccgagcgtggccgcctggcaggtatggaacgagcccaacctgatcggcttctggcggcccaaggccgacccggaaggctacgccaagctgctccaggccagcaccatcgccctgcgcatggtcgacccggagaagccggtggtttccgccggcatggccttcttcagcgagatgcccgacggccgcaccatgttcgacgccctcggccacctgggcgtggagagcctcggcaccatcgccacctaccacccctatacccagttgccggaaggcaactacccgtggaacctggacttcgtctcccacgccaaccagatcaaccgcgccctgcgcaacgccggcgtgccggcgatctggagcaccgagtggggctggtcggcctacaaggggccgaaggagttgcaggacatcattggcgtcgaaggccaggccgactacgtgctgcgtcgcctggcgctgatgagtgcgctggactacgaccggatcttcctcttcaccctcagcgatctcgaccagcgcgccagcgtgcgcgaccgcgactacggcctgctcgacctggacgccaaccccaagccggtctacctggccctgcaacgcttcctcaaggtcaccgggccgaagctgcgcccggccgacccgccggtcaccgaggacctgcccgacggttccttcagcatcggctggacccgcgaggacggtcgcaacgtctggctgttctggtcggcccgcggcggcaacgtgcgcctgccgaagctcaaggaggccaccctgcacgatccgctcagcggcaaggtcacgcccttgagcggcagcgacggcctggaagtcccggtgaagtccagcctgcagatgctggtctgggagtga

SEQ ID NO: 6 - protein, 412 aa, Artificial sequence
  Synthesized polypeptide
EIQVLKAPRAVVWKDFLGVNAQFLWFSPERYNKQIDRLQDLGLEWVRLDLHWDRLETAEDQYQLASLDQLVKDLEARQLKSVFYLVGSARFITTAPFYSPFQDQYPPRDPEVFARRMAMLSQRYPSVAAWQVWNEPNLIGFWRPKADPEGYAKLLQASTIALRMVDPEKPVVSAGMAFFSEMPDGRTMFDALGHLGVESLGTIATYHPYTQLPEGNYPWNLDFVSHANQINRALRNAGVPAIWSTEWGWSAYKGPKELQDIIGVEGQADYVLRRLALMSALDYDRIFLFTLSDLDQRASVRDRDYGLLDLDANPKPVYLALQRFLKVTGPKLRPADPPVTEDLPDGSFSIGWTREDGRNVWLFWSARGGNVRLPKLKEATLHDPLSGKVTPLSGSDGLEVPVKSSLQMLVWE

SEQ ID NO: 7 - DNA, 24 bp, Artificial sequence
  Synthetic sequence
agaattgaggttctcgtcttcagg

SEQ ID NO: 8 - DNA, 45 bp, Artificial sequence
  Synthetic sequence
gatctggctggcgctcttctgcatgtgaggcgatgatcgataagc

SEQ ID NO: 9 - DNA, 45 bp, Artificial sequence
  Synthetic sequence
gcttatcgatcatcgcctcacatgcagaagagcgccagccagatc

SEQ ID NO: 10 - DNA, 30 bp, Artificial sequence
  Synthetic sequence
gtcaagcttctgctggtctgtagcgaggac

SEQ ID NO: 11 - DNA, 27 bp, Artificial sequence
  Synthetic sequence
ggaattcgatggtggtgatgacgatgc

SEQ ID NO: 12 - DNA, 32 bp, Artificial sequence
  Synthetic sequence
ctccgttccttgtgaggcgatgatcgataagc

SEQ ID NO: 13 - DNA, 32 bp, Artificial sequence
  Synthetic sequence
catcgcctcacaaggaacggagtctcatgagc

SEQ ID NO: 14 - DNA, 33 bp, Artificial sequence
  Synthetic sequence
cgctcttctgcatctgcagcgaccgcgattgtg

SEQ ID NO: 15 - DNA, 35 bp, Artificial sequence
  Synthetic sequence
ggtcgctgcagatgcagaagagcgccagccagatc

SEQ ID NO: 16 - DNA, 28 bp, Artificial sequence
  Synthetic sequence
cgggatccgtgcgaccctcaaccgtttc

SEQ ID NO: 17 - DNA, 27 bp, Artificial sequence
  Synthetic sequence
ggaattcgatggtggtgatgacgatgc

SEQ ID NO: 18 - DNA, 32 bp, Artificial sequence
  Synthetic sequence
cgaaggtgccatgagactccgttccttgtgag

SEQ ID NO: 19 - DNA, 31 bp, Artificial sequence
  Synthetic sequence
gaacggagtctcatggcaccttcggaagcgc

SEQ ID NO: 20 - DNA, 33 bp, Artificial sequence; identical to SEQ ID NO: 14
  Synthetic sequence

SEQ ID NO: 21 - DNA, 35 bp, Artificial sequence; identical to SEQ ID NO: 15
  Synthetic sequence

SEQ ID NO: 22 - DNA, 28 bp, Artificial sequence
  Synthetic sequence
cgggatccgtgcgaccctcaaccgtttc

SEQ ID NO: 23 - DNA, 26 bp, Artificial sequence
  Synthetic sequence
ggaattccgggaagaatttggtgatg

SEQ ID NO: 24 - DNA, 28 bp, Artificial sequence
  Synthetic sequence
cgggatccttacttgatgaagtcgtgac

SEQ ID NO: 25 - DNA, 40 bp, Artificial sequence
  Synthetic oligonucleotide
tatgacatgattacgaattccgtcgagaagatccattcgc

SEQ ID NO: 26 - DNA, 36 bp, Artificial sequence
  Synthetic sequence
gcatagtcggtctagaggcgatcaatgcgagagaag

SEQ ID NO: 27 - DNA, 36 bp, Artificial sequence
  Synthetic sequence
attgatcgcctctagaccgactatgcgcagttcttc

SEQ ID NO: 28 - DNA, 40 bp, Artificial sequence
  Synthetic sequence
acgacggccagtgccaagcttcaatctcgtccagtgcagc